(19) United States
(12) Patent Application Publication
(10) Pub. No.: US 2026/0102125 A1
(43) Pub. Date: Apr. 16, 2026

(54) CALIBRATION OF COUNTERPRESSURES USING FORCE SENSOR

(71) Applicant: QUALCOMM Incorporated, San Diego, CA (US)

(72) Inventors: Samuel Jon EUBANKS, San Diego, CA (US); Emily Kathryn BROOKS, Amherst, NY (US); John Keith SCHNEIDER, Williamsville, NY (US); Evan Michael BRELOFF, Kenmore, NY (US); Courtney WOLFF, Cheektowaga, NY (US); Wenhan ZHENG, Amherst, NY (US); Hrishikesh Vijaykumar PANCHAWAGH, Cupertino, CA (US); Stephen Michael GOJEVIC, Lockport, NY (US)

(21) Appl. No.: 18/916,457

(22) Filed: Oct. 15, 2024

(51) Int. Cl. A61B 5/00; A61B 5/021; A61B 7/04

(52) U.S. Cl. CPC A61B 5/7278; A61B 5/0095; A61B 5/021; A61B 5/6843; A61B 7/04; A61B 2560/0223

(57) ABSTRACT

Methods, apparatus, and systems for calibration of counterpressures using a force sensor and/or estimation of a physiological parameter of a user (such as blood pressure) are disclosed. In some embodiments, a user device may include: a signal source system; a sensor system; an interface portion; a force sensor system, the force sensor system configured to measure an external pressure applied to the interface portion during contact by the user; and a control system configured to: obtain calibration data correlating one or more external pressure values to one or more calibration factor values; determine, using the calibration data, a calibration factor based on the external pressure applied to the interface portion during the contact by the user; and estimate a physiological parameter based on the calibration factor, and one or more physical characteristics of the target object of the user determined based on the signals detected by the sensor system.

### BACKGROUND

### 1. Field of Disclosure

The present disclosure relates generally to devices and systems using biometric sensors, including non-invasive biometric sensors.

### 2. Description of Related Art

A variety of different sensing technologies and algorithms are being implemented in devices for various biometric and biomedical applications, including health and wellness monitoring. This push is partly a result of the limitations in the usability of traditional measuring devices for continuous, noninvasive and/or ambulatory monitoring. Some such devices are, or include, photoacoustic and pulse echo ultrasound (PeU) sensors. Although some previously deployed devices can provide acceptable results, improved detection devices and systems would be desirable

### BRIEF SUMMARY

In some aspects of the present disclosure, a user device is disclosed. In some embodiments, the user device may include: a signal source system configured to emit optical signals, acoustic signals, electromagnetic signals, or a combination thereof toward a target object; a sensor system configured to detect signals generated from the emitted optical signals, the emitted acoustic signals, the emitted electromagnetic signals, or the combination thereof incident on the target object; an interface portion contactable by a user; and a force sensor system configured to measure an external pressure applied to the interface portion during contact by the user.

In some embodiments, the user device may further include a control system configured to: obtain calibration data correlating one or more external pressure values to one or more calibration factor values; determine, using the calibration data, a calibration factor based on the external pressure applied to the interface portion during the contact by the user; and estimate a physiological parameter based on the calibration factor, and one or more physical characteristics of the target object of the user determined based on the signals detected by the sensor system.

In some implementations, the estimated physiological parameter may include a blood pressure of the user.

In some implementations, the control system may be further configured to, to obtain the calibration data: apply a plurality of external pressure values; measure one or more physical characteristics of the target object at each of the plurality of external pressure values; determine the one or more calibration factor values, each based on the one or more physical characteristics of the target object; and determine a relationship between the plurality of external pressure values and the one or more calibration factor values.

In some implementations, the target object may include a blood vessel of the user.

In some implementations, the signal source system may include a light source system configured to generate and emit the optical signals toward the blood vessel of the user; and the sensor system may include a photoacoustic sensor system configured to obtain photoacoustic signals generated from the emitted optical signals incident on the blood vessel of the user, a pulse echo ultrasound (PeU) sensor system configured to obtain ultrasonic waves generated from the emitted acoustic signals incident on the blood vessel of the user, or a combination thereof.

In some aspects of the present disclosure, a system configured to estimate a physiological parameter is disclosed. In some embodiments, the system may include: a housing comprising: a signal source system configured to emit optical signals, acoustic signals, electromagnetic signals, or a combination thereof toward a target object; a sensor system configured to detect acoustic signals generated from the emitted optical signals, the emitted acoustic signals, the emitted electromagnetic signals, or the combination thereof incident on the target object; and an interface portion contactable by a user; and a force sensor system disposed externally to the housing, the force sensor system configured to measure an external pressure applied during contact by the user.

In some embodiments, the system may further include a control system configured to: obtain calibration data correlating one or more external pressure values to one or more calibration factor values; determine, using the calibration data, a calibration factor based at least on the external pressure during the contact by the user; and estimate a physiological parameter based at least on the calibration factor and one or more physical characteristics of the target object of the user determined based on the acoustic signals detected by the sensor system.

In some implementations, the force sensor system may be disposed externally to the housing such that the housing is disposed between the force sensor system and the target object, or such that the target object is disposed between the housing and the force sensor system.

In some aspects of the present disclosure, a method of estimating a physiological parameter using a user device is disclosed. In some embodiments, the method may include: obtaining calibration data correlating one or more external pressure values to one or more calibration factor values, and obtaining one or more signals generated from a target object of a user; during operation of the user device, obtaining, via a force sensor associated with the user device, a measurement of an external pressure applied to the target object of the user; determining, using the calibration data, a calibration factor based on the measurement of the external pressure applied to the target object of the user; and estimating the physiological parameter based on the calibration factor and one or more physical characteristics of the target object, the one or more physical characteristics of the target object determined based on the one or more signals generated from the target object.

In some implementations, the estimated physiological parameter may include a blood pressure of the user.

In some implementations, the force sensor associated with the user device may be disposed internally to the user device and configured to detect force associated with the external pressure via an interface of the user device. In some implementations, the force sensor associated with the user device may be disposed externally to the user device such that the user device is disposed between the force sensor and the target object, or such that the target object is disposed between the user device and the force sensor.

In some implementations, the obtaining of the calibration data may include: applying a plurality of external pressure values; measuring one or more physical characteristics of the target object at each of the plurality of external pressure values; determining the one or more calibration factor values, each based on the one or more physical characteristics of the target object; and determining a relationship between the plurality of external pressure values and the one or more calibration factor values.

This summary is neither intended to identify key or essential features of the claimed subject matter, nor is it intended to be used in isolation to determine the scope of the claimed subject matter. The subject matter should be understood by reference to appropriate portions of the entire specification of this disclosure, any or all drawings, and each claim. The foregoing, together with other features and examples, will be described in more detail below in the following specification, claims, and accompanying drawings.

### BRIEF DESCRIPTION OF THE DRAWINGS

FIG. 1 shows an example of a blood pressure monitoring device based on photoacoustic plethysmography, which may be referred to herein as PAPG.

FIG. 2 shows an example of a blood pressure monitoring device based on photoplethysmography (PPG).

FIG. 3 shows examples of heart rate waveform (HRW) features that may be extracted according to some implementations.

FIG. 4 shows a cross-sectional side view of a diagrammatic representation of a portion of an artery through which a pulse is propagating.

FIG. 5A shows an example monitoring device designed to be worn around a wrist according to some implementations

FIG. 5B shows an example monitoring device designed to be worn on a finger according to some implementations.

FIG. 5C shows an example monitoring device designed to reside on an earbud according to some implementations.

FIG. 6 is a block diagram that shows example components of a sensor apparatus according to some disclosed embodiments.

FIGS. 7A and 7B illustrate cross-sectional views of an example configuration of a sensor apparatus having an internal force sensor, according to some disclosed embodiments.

FIGS. 8A-8C illustrate views of simplified representations of an example configuration of a sensor apparatus having an internal force sensor, according to some disclosed embodiments.

FIGS. 9A and 9B illustrate cross-sectional views of another example configuration of a sensor apparatus having an internal force sensor, according to some disclosed embodiments.

FIG. 10 illustrates a cross-sectional view of an example configuration of a sensor apparatus having an external force sensor, according to some disclosed embodiments.

FIG. 11 illustrates a cross-sectional view of another example configuration of a sensor apparatus having an external force sensor, according to some disclosed embodiments.

FIG. 12 is an example graph of external pressure as a function of calibration factor (α), representative of calibration data useful with some disclosed embodiments.

FIG. 13 is a block diagram that shows a flow diagram for determining a physiological parameter using measurements by a sensor apparatus and calibration data, which can be utilized in embodiments as described herein.

FIG. 14 is a flow diagram of estimating a physiological parameter using a user device, according to some embodiments.

Like reference symbols in the various drawings indicate like elements, in accordance with certain example implementations. In addition, multiple instances of an element may be indicated by following a first number for the element with a letter or a hyphen and a second number. For example, multiple instances of an element 110 may be indicated as 110-1, 110-2, 110-3 etc. or as 110a, 110b, 110c, etc. When referring to such an element using only the first number, any instance of the element is to be understood (e.g., element 110 in the previous example would refer to elements 110-1, 110-2, and 110-3 or to elements 110a, 110b, and 110c).

### DETAILED DESCRIPTION

The following description is directed to certain implementations for the purposes of describing various aspects of this disclosure. However, a person having ordinary skill in the art will readily recognize that the teachings herein can be applied in a multitude of different ways. Some of the concepts and examples provided in this disclosure are especially applicable to user sensing applications. For example, non-invasive (e.g., cuffless) biometric sensing can be performed using the disclosed embodiments. However, some implementations also may be applicable to other types of sensing applications including biometric sensing, as well as to various other systems. The described implementations may be implemented in any device, apparatus, or system that includes an apparatus as disclosed herein. In addition, it is contemplated that the described implementations may be included in or associated with a variety of electronic devices (which may also be referred to herein simply as “devices” or a “device”) such as, but not limited to, mobile telephones, multimedia Internet-enabled cellular telephones, mobile television receivers, wireless devices, smartphones, smart cards, tablets, wearable devices such as bracelets, armbands, wristbands, watches, smartwatches, rings, headbands, patches, chest bands, anklets, etc., Bluetooth® devices, personal data assistants (PDAs), wireless electronic mail receivers, handheld or portable computers, netbooks, notebooks, smartbooks, printers, copiers, scanners, facsimile devices, global positioning system (GPS) receivers or navigators, cameras, digital media players, game consoles, wrist watches, clocks, calculators, television monitors, flat panel displays, electronic reading devices (e.g., e-readers), mobile health devices, computer monitors, auto displays (including odometer and speedometer displays, dashboard displays, etc.), cockpit controls and/or displays, camera view displays (such as the display of a rear view camera in a vehicle), architectural structures, microwaves, refrigerators, stereo systems, cassette recorders or players, DVD players, CD players, VCRs, radios, portable memory chips, washers, dryers, washer/dryers, parking meters, automobile doors, Internet of Things (IoT) devices, palm scanners, or point-of-sale (POS) terminals. Thus, the teachings are not intended to be limited to the specific implementations depicted and described with reference to the drawings; rather, the teachings have wide applicability as will be readily apparent to persons having ordinary skill in the art.

There is a strong need for accurate, non-invasive, continuous monitoring devices for both clinical and consumer applications, e.g., for measuring physiological parameters such as blood pressure of a user. In particular, non-invasive, contactless monitoring of blood pressure using a user-wearable device is desirable. Continuous blood pressure monitoring opens avenues for efficient and effective diagnosis and treatment of cardiovascular conditions (e.g., hypertension), cardiovascular event detection, and stress monitoring. It would also allow daily spot checks of cardiovascular conditions including blood pressure, as well as overnight sleep monitoring. Positive user experience during overnight sleep monitoring is desirable. For example, there should be minimal discomfort to the user during operation of the wearable device, including during sleep. Hence, a device that does not apply significant external pressure to the user is desirable.

Contactless sensing mechanisms that allow collection of biometrics and measurement of physiological characteristics such as pulse wave velocity (PWV) of a blood vessel, and arterial measurements such as diameter, cross-sectional area, volume, and/or distension, could enable the above approaches. PWV and compliance are relevant characteristics that are a function of the arterial wall stiffness and tension, blood density, body posture, blood pressure, and more. It would thus be valuable for blood pressure estimation to obtain such characteristics with accuracy and convenience.

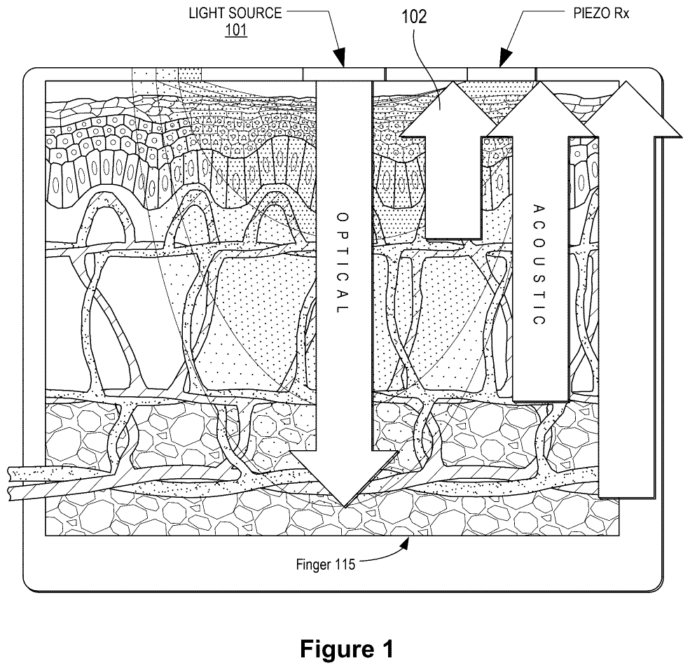

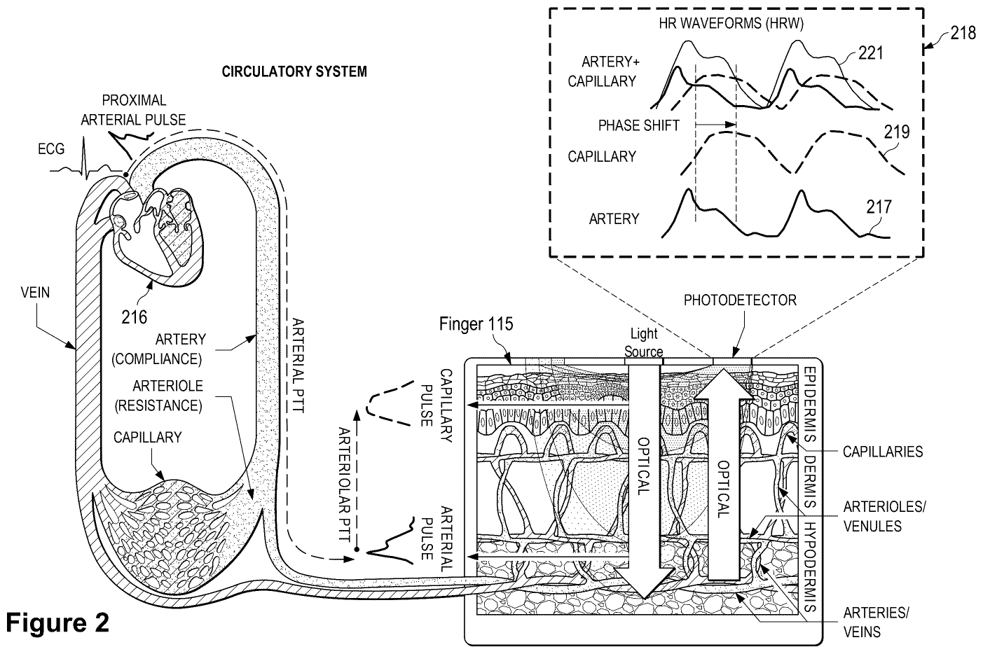

To these ends, various sensing modalities, including methods to generate an acoustic response from a material or target object (e.g., body part such as a finger, or portion of tissue such as a blood vessel) may be used with sensing mechanisms for such non-invasive sensing and monitoring. For example, photoacoustics (e.g., photoacoustic plethysmography or PAPG) and pulse echo ultrasound (PeU) are being pursued as methods of obtaining non-invasive and cuffless measurements of blood pressure and/or other physiological information. Sec discussion of FIG. 1 below. Another example sensing modality is photoplethysmography (PPG), which can be used to monitor biological parameters in a non-invasive way by transmitting light and receiving reflected light from a target object. See discussion of FIG. 2 below. Another example sensing modality may include an infrared (IR) light-emitting diode (LED) and an IR sensor to detect blood volume. Another example sensing modality may involve a radio frequency (RF) transmitter (emitting frequencies in the MHz-GHz range) and an RF sensor configured to detect changes in electrical impedance caused by blood flow.

In some embodiments, sensors required to perform these measurements can be used with a sensing apparatus or device having one of various form factors. In some configurations, cuffless devices may be used. For example, a probe configuration with a sensing area that contacts a user's skin may be implemented as a handheld operating device and/or a benchtop device. Other non-invasive form factors can be used as well, such as a ring, wristband, or other wearable devices.

It is useful that the applied external pressure from a device contacting a surface, e.g., of skin or tissue of a user's body part (also known as a counterpressure in the context of the present disclosure) be known and/or controlled to obtain accurate measurements (e.g., of blood pressure, other physiological parameters or characteristics). Ideally, the counterpressure is zero (representing no contact) for estimating physiological information such as blood pressure. However, in real measurement scenarios, there is some level of contact and thus pressure on a body part, distorting internal tissue including blood vessels and thereby affecting physical characteristics thereof (e.g., diameter, distension, cross-sectional area).

Put another way, it would be helpful to obtain measurements and data useful blood pressure estimation at a known applied counterpressure. There are currently limitations when obtaining this data. Current and proposed methods of estimating blood pressure may also require data from several different intentionally applied pressure steps. Typically, acquisition of this data lasts anywhere from 5 to 20 seconds using a cuffed device (e.g., a sphygmomanometer or automatic sphygmomanometer), during which the user is asked to remain still (e.g., as various cuff pressures are sequentially applied to the user over the 5-20 seconds). Several external applied pressures may be used to characterize the target object (e.g., blood vessel), such as how the diameter, distension, and cross-sectional area of an artery changes with pressure. Any variations in the applied pressure during data collection should also be measured. Moreover, a counterpressure changes the diameter, distension, and cross-sectional area of the artery being imaged, which affects measurements.

Knowledge of the applied counterpressure is especially salient information in cases where test data is being compared to calibration data collected at a previous point in time. There are consistency issues not only with ad hoc measurements but also day-to-day fluctuations in how users places their body part (e.g., hand or wrist) with respect to the sensing device or system, which may unintentionally result in changing the counterpressure being applied, impacting the relationship between the test and calibration data.

As such, it is desirable to have the ability to both measure and control the applied counterpressure.

In some embodiments, a force sensor (e.g., capacitive or resistive load cell) used with the cuffless device can provide the necessary information on externally applied force or pressure to estimate physiological parameters of the user, such as blood pressure. In some configurations, the force sensor may be disposed internally to (inside) the sensing device. In some configurations, the force sensor may be disposed externally to (outside) the sensing device, e.g., when in a benchtop configuration.

In some embodiments described in the present disclosure, the force information may be used with calibration data obtained to derive information that can be used to estimate a physiological parameter such as blood pressure. Relationships between calibration information and physical characteristics of a blood vessel, as will be described below, allow for such estimation.

In some implementations, machine learning can be used to train a machine learning or artificial intelligence model that can predict a physical characteristic of a target object (e.g., PWV of blood vessel) or physiological parameter of the user (e.g., blood pressure). As will be discussed further, calibration data can also be obtained using machine learning approaches.

Particular implementations of the subject matter described in this disclosure can be implemented to realize one or more of the following potential advantages. Using calibration data in conjunction with direct measurement of force or pressure applied to the sensing device allows estimation of user parameters (e.g., blood pressure) without using cuffs or lengthy data collection processes, e.g., ramping up cuff pressure to fully occlude the artery and then ramping down the cuff pressure to zero. Calibration data need only be obtained once (or more if recalibration is needed) and can be specific to the user, providing a customized estimation of parameters (e.g., blood pressure) catered to the individual user's unique physiology. In addition, implementation of a force sensor with the sensing device enables a simplified yet accurate measurement of user parameters. Such an approach would allow a user to wear a cuffless sensing device comfortably to enable continuous monitoring of biometrics. Further, low-power and low-footprint modalities such as PAPG and PeU can be useful for non-invasive blood pressure detection and monitoring.

Additional details will follow after an initial description of relevant systems and technologies.

FIG. 1 shows an example of a blood pressure monitoring device based on photoacoustic plethysmography, which is referred to herein as PAPG. FIG. 1 shows the same examples of arteries, veins, arterioles, venules and capillaries inside a body part, which is a finger 115 in this example. In some examples, the light source shown in FIG. 1 may be coupled to a light source system (not shown) that is disposed remotely from the body part (e.g., finger 115). In some implementations, the light source may be an opening of an optical fiber or other waveguide. Such an opening may also be connected to an opening of an interface that is contactable with the body part. In some embodiments, the light source system may include one or more LEDs, one or more laser diodes, etc. In this example, the light source has transmitted light (in some examples, green, red, infrared, and/or near-infrared (NIR) light) that has penetrated the tissues of the finger 115 in an illuminated zone.

In the example shown in FIG. 1, blood vessels (and components of the blood itself) are heated by the incident light from the light source and are emitting acoustic waves 102. In this example, the emitted acoustic waves 102 include ultrasonic waves. According to this implementation, the acoustic wave emissions 102 are being detected by an ultrasonic receiver, which is a piezoelectric receiver in this example. Photoacoustic emissions 102 from the illuminated tissues, detected by the piezoelectric receiver, may be used to detect volumetric changes in the blood of the illuminated zone of the finger 115 that correspond to physiological data within the illuminated tissues of finger 115, such as heart rate waveforms. Although some of the tissue areas shown to be illuminated are offset from those shown to be producing photoacoustic emissions 102, this is merely for illustrative convenience. It will be appreciated that that the illuminated tissues will actually be those producing photoacoustic emissions. Moreover, it will be appreciated that the maximum levels of photoacoustic emissions will often be produced along the same axis as the maximum levels of illumination.

One important difference between an optical technique such as a photoplethysmography (PPG)-based system the PAPG-based method of FIG. 1 is that the acoustic waves shown in FIG. 1 travel much more slowly than the reflected light waves involved in PPG. Accordingly, depth discrimination based on the arrival times of the acoustic waves shown in FIG. 1 is possible, whereas depth discrimination based on the arrival times of the light waves in PPG may not be possible. This depth discrimination allows some disclosed implementations to isolate acoustic waves received from the different blood vessels.

According to some such examples, such depth discrimination allows artery heart rate waveforms to be distinguished from vein heart rate waveforms and other heart rate waveforms. Therefore, blood pressure estimation based on depth-discriminated PAPG methods can be substantially more accurate than blood pressure estimation based on PPG-based methods.

FIG. 2 shows an example of a blood pressure monitoring device based on photoplethysmography (PPG). FIG. 2 shows examples of arteries, veins, arterioles, venules and capillaries of a circulatory system, including those inside a finger 115. In the example shown in FIG. 2, an electrocardiogram (ECG) sensor has detected a proximal arterial pulse near the heart 216. Some examples are described below of measurement of the arterial pulse transit time (PTT) according to arterial pulses measured by two sensors, one of which may be an electrocardiogram sensor in some implementations.

According to the example shown in FIG. 2, a light source that includes one or more lasers or light-emitting diodes (LEDs) has transmitted light (in some examples, green, red, infrared, and/or near-infrared (NIR) light) that has penetrated the tissues of the finger 115 in an illuminated zone. Reflections from these tissues, detected by a photodetector, may be used to detect volumetric changes in the blood of the illuminated zone of the finger 115 that correspond to heart rate waveforms.

As shown in the heart rate waveform graphs 218 of FIG. 2, the capillary heart rate waveform 219 is differently shaped and phase-shifted relative to the artery heart rate waveform 217. In this simple example, the detected heart rate waveform 221 is a combination of the capillary heart rate waveform 219 and the artery heart rate waveform 217. In some instances, the responses of one or more other blood vessels may also be part of the heart rate waveform 221 detected by a PPG-based blood pressure monitoring device.

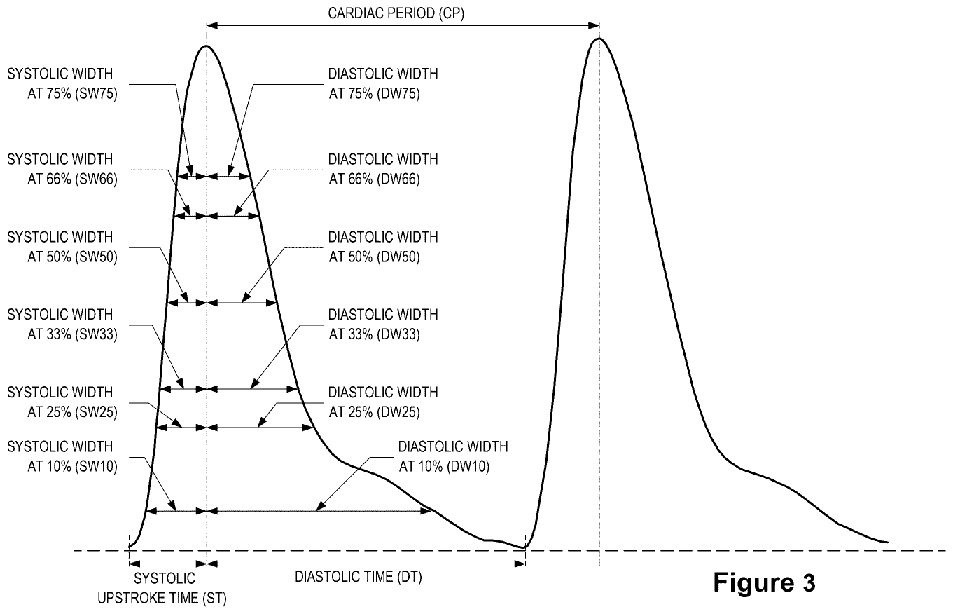

FIG. 3 shows examples of heart rate waveform (HRW) features that may be extracted according to some implementations. The horizontal axis of FIG. 3 represents time and the vertical axis represents signal amplitude. The cardiac period is indicated by the time between adjacent peaks of the HRW. The systolic and diastolic time intervals are indicated below the horizontal axis. During the systolic phase of the cardiac cycle, as a pulse propagates through a particular location along an artery, the arterial walls expand according to the pulse waveform and the elastic properties of the arterial walls. Along with the expansion is a corresponding increase in the volume of blood at the particular location or region, and with the increase in volume of blood an associated change in one or more characteristics in the region. Conversely, during the diastolic phase of the cardiac cycle, the blood pressure in the arteries decreases and the arterial walls contract. Along with the contraction is a corresponding decrease in the volume of blood at the particular location, and with the decrease in volume of blood an associated change in the one or more characteristics in the region.

The HRW features that are illustrated in FIG. 3 pertain to the width of the systolic and/or diastolic portions of the HRW curve at various “heights,” which are indicated by a percentage of the maximum amplitude. For example, the SW50 feature is the width of the systolic portion of the HRW curve at a “height” of 50% of the maximum amplitude. In some implementations, the HRW features used for blood pressure estimation may include some or all of the SW10, SW25, SW33, SW50, SW66, SW75, DW10, DW25, DW33, DW50, DW66 and DW75 HRW features. In other implementations, additional HRW features may be used for blood pressure estimation. Such additional HRW features may, in some instances, include the sum and ratio of the SW and DW at one or more “heights,” e.g., (DW75+SW75), DW75/SW75, (DW66+SW66), DW66/SW66, (DW50+SW50), DW50/SW50, (DW33+SW33), DW33/SW33, (DW25+SW25), DW25/SW25 and/or (DW10+SW10), DW10/SW10. Other implementations may use yet other HRW features for blood pressure estimation. Such additional HRW features may, in some instances, include sums, differences, ratios and/or other operations based on more than one “height,” such as (DW75+SW75)/(DW50+SW50), (DW50+SW50/(DW10+SW10), etc.

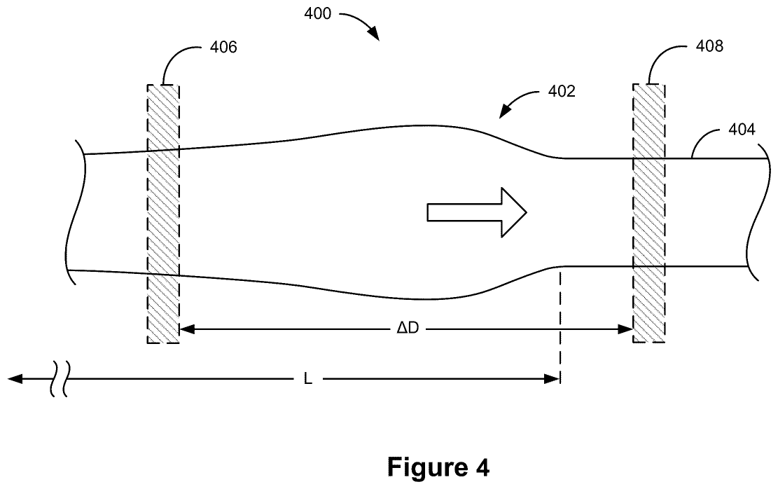

FIG. 4 shows a cross-sectional side view of a diagrammatic representation of a portion of an artery 400 through which a pulse 402 is propagating. The block arrow in FIG. 4 shows the direction of blood flow and pulse propagation. As diagrammatically shown, the propagating pulse 402 causes strain in the arterial walls 404, which is manifested in the form of an enlargement in the diameter (and consequently the cross-sectional area) of the arterial walls-referred to as “distension.” The spatial length L of an actual propagating pulse along an artery (along the direction of blood flow) is typically comparable to the length of a limb, such as the distance from a subject's shoulder to the subject's wrist or finger, and is generally less than one meter (m). However, the length L of a propagating pulse can vary considerably from subject to subject, and for a given subject, can vary significantly over durations of time depending on various factors. The spatial length L of a pulse will generally decrease with increasing distance from the heart until the pulse reaches capillaries.

As described above, some particular implementations relate to devices, systems and methods for estimating blood pressure or other cardiovascular characteristics based on estimates of an arterial distension waveform. The terms “estimating,” “measuring,” “calculating,” “inferring,” “deducing,” “evaluating,” “determining” and “monitoring” may be used interchangeably herein where appropriate unless otherwise indicated. Similarly, derivations from the roots of these terms also are used interchangeably where appropriate; for example, the terms “estimate,” “measurement,” “calculation,” “inference” and “determination” also are used interchangeably herein. In some implementations, the pulse wave velocity (PWV) of a propagating pulse may be estimated by measuring the pulse transit time (PTT) of the pulse as it propagates from a first physical location along an artery to another more distal second physical location along the artery. However, either version of the PTT may be used for the purpose of blood pressure estimation. Assuming that the physical distance ΔD between the first and the second physical locations is ascertainable, the PWV can be estimated as the quotient of the physical spatial distance ΔD traveled by the pulse divided by the time (PTT) the pulse takes in traversing the physical spatial distance ΔD. Generally, a first sensor positioned at the first physical location is used to determine a starting time (also referred to herein as a “first temporal location”) at which point the pulse arrives at or propagates through the first physical location. A second sensor at the second physical location is used to determine an ending time (also referred to herein as a “second temporal location”) at which point the pulse arrives at or propagates through the second physical location and continues through the remainder of the arterial branch. In such examples, the PTT represents the temporal distance (or time difference) between the first and the second temporal locations (the starting and the ending times).

The fact that measurements of the arterial distension waveform are performed at two different physical locations implies that the estimated PWV inevitably represents an average over the entire path distance ΔD through which the pulse propagates between the first physical location and the second physical location. More specifically, the PWV generally depends on a number of factors including the density of the blood ρ, the stiffness E of the arterial wall (or inversely the elasticity), the arterial diameter, the thickness of the arterial wall, and the blood pressure. Because both the arterial wall elasticity and baseline resting diameter (for example, the diameter at the end of the ventricular diastole period) vary significantly throughout the arterial system, PWV estimates obtained from PTT measurements are inherently average values (averaged over the entire path length ΔD between the two locations where the measurements are performed).

In traditional methods for obtaining PWV, the starting time of the pulse has been obtained at the heart using an electrocardiogram (ECG) sensor, which detects electrical signals from the heart. For example, the starting time can be estimated based on the QRS complex—an electrical signal characteristic of the electrical stimulation of the heart ventricles. In such approaches, the ending time of the pulse is typically obtained using a different sensor positioned at a second location (for example, a finger). As a person having ordinary skill in the art will appreciate, there are numerous arterial discontinuities, branches, and variations along the entire path length from the heart to the finger. The PWV can change by as much as or more than an order of magnitude along various stretches of the entire path length from the heart to the finger. As such, PWV estimates based on such long path lengths are unreliable.

In various implementations described herein, PTT estimates are obtained based on measurements (also referred to as “arterial distension data” or more generally as “sensor data”) associated with an arterial distension signal obtained by each of a first arterial distension sensor 406 and a second arterial distension sensor 408 proximate first and second physical locations, respectively, along an artery of interest. In some particular implementations, the first arterial distension sensor 406 and the second arterial distension sensor 408 are advantageously positioned proximate first and second physical locations between which arterial properties of the artery of interest, such as wall elasticity and diameter, can be considered or assumed to be relatively constant. In this way, the PWV calculated based on the PTT estimate is more representative of the actual PWV along the particular segment of the artery. In turn, the blood pressure P estimated based on the PWV is more representative of the true blood pressure. In some implementations, the magnitude of the distance ΔD of separation between the first arterial distension sensor 406 and the second arterial distension sensor 408 (and consequently the distance between the first and the second locations along the artery) can be in the range of about 1 centimeter (cm) to tens of centimeters—long enough to distinguish the arrival of the pulse at the first physical location from the arrival of the pulse at the second physical location, but close enough to provide sufficient assurance of arterial consistency. In some specific implementations, the distance ΔD between the first and the second arterial distension sensors 406 and 408 can be in the range of about 1 cm to about 30 cm, and in some implementations, less than or equal to about 20 cm, and in some implementations, less than or equal to about 10 cm, and in some specific implementations less than or equal to about 5 cm. In some other implementations, the distance ΔD between the first and the second arterial distension sensors 406 and 408 can be less than or equal to 1 cm, for example, about 0.1 cm, about 0.25 cm, about 0.5 cm or about 0.75 cm. By way of reference, a typical PWV can be about 15 meters per second (m/s). Using a monitoring device in which the first and the second arterial distension sensors 406 and 408 are separated by a distance of about 5 cm, and assuming a PWV of about 15 m/s implies a PTT of approximately 3.3 milliseconds (ms).

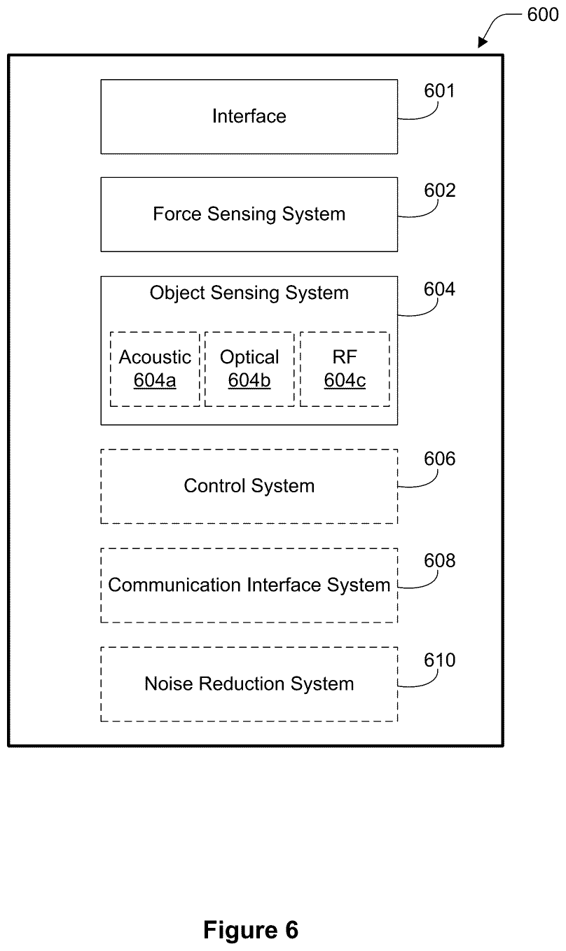

The value of the magnitude of the distance ΔD between the first and the second arterial distension sensors 406 and 408, respectively, can be preprogrammed into a memory within a monitoring device that incorporates the sensors (for example, such as a memory of, or a memory configured for communication with a control system such as 706 that is described below with reference to FIG. 7). As will be appreciated by a person of ordinary skill in the art, the spatial length L of a pulse can be greater than the distance ΔD from the first arterial distension sensor 406 to the second arterial distension sensor 408 in such implementations. As such, although the diagrammatic pulse 402 shown in FIG. 4 is shown as having a spatial length L comparable to the distance between the first arterial distension sensor 406 and the second arterial distension sensor 408, in actuality each pulse can typically have a spatial length L that is greater and even much greater than (for example, about an order of magnitude or more than) the distance ΔD between the first and the second arterial distension sensors 406 and 408.

In some implementations of the monitoring devices disclosed herein, both the first arterial distension sensor 406 and the second arterial distension sensor 408 are sensors of the same sensor type. In some such implementations, the first arterial distension sensor 406 and the second arterial distension sensor 408 are identical sensors. In such implementations, each of the first arterial distension sensor 406 and the second arterial distension sensor 408 utilizes the same sensor technology with the same sensitivity to the arterial distension signal caused by the propagating pulses, and has the same time delays and sampling characteristics. In some implementations, each of the first arterial distension sensor 406 and the second arterial distension sensor 408 is configured for photoacoustic plethysmography (PAPG) sensing, e.g., as disclosed elsewhere herein. Some such implementations include a light source system and two or more ultrasonic receivers. In some implementations, each of the first arterial distension sensor 406 and the second arterial distension sensor 408 is configured for ultrasound sensing via the transmission of ultrasonic signals and the receipt of corresponding reflections. In some alternative implementations, each of the first arterial distension sensor 406 and the second arterial distension sensor 408 may be configured for impedance plethysmography (IPG) sensing, also referred to in biomedical contexts as bioimpedance sensing. In various implementations, whatever types of sensors are utilized, each of the first and the second arterial distension sensors 406 and 408 broadly functions to capture and provide arterial distension data indicative of an arterial distension signal resulting from the propagation of pulses through a portion of the artery proximate to which the respective sensor is positioned. For example, the arterial distension data can be provided from the sensor to a processor in the form of voltage signal generated or received by the sensor based on an ultrasonic signal or an impedance signal sensed by the respective sensor.

As described above, during the systolic phase of the cardiac cycle, as a pulse propagates through a particular location along an artery, the arterial walls expand according to the pulse waveform and the elastic properties of the arterial walls. Along with the expansion is a corresponding increase in the volume of blood at the particular location or region, and with the increase in volume of blood an associated change in one or more characteristics in the region. Conversely, during the diastolic phase of the cardiac cycle, the blood pressure in the arteries decreases and the arterial walls contract. Along with the contraction is a corresponding decrease in the volume of blood at the particular location, and with the decrease in volume of blood an associated change in the one or more characteristics in the region.

In the context of bioimpedance sensing (or impedance plethysmography), the blood in the arteries has a greater electrical conductivity than that of the surrounding or adjacent skin, muscle, fat, tendons, ligaments, bone, lymph or other tissues. The susceptance (and thus the permittivity) of blood also is different from the susceptances (and permittivities) of the other types of surrounding or nearby tissues. As a pulse propagates through a particular location, the corresponding increase in the volume of blood results in an increase in the electrical conductivity at the particular location (and more generally an increase in the admittance, or equivalently a decrease in the impedance). Conversely, during the diastolic phase of the cardiac cycle, the corresponding decrease in the volume of blood results in an increase in the electrical resistivity at the particular location (and more generally an increase in the impedance, or equivalently a decrease in the admittance).

A bioimpedance sensor generally functions by applying an electrical excitation signal at an excitation carrier frequency to a region of interest via two or more input electrodes, and detecting an output signal (or output signals) via two or more output electrodes. In some more specific implementations, the electrical excitation signal is an electrical current signal injected into the region of interest via the input electrodes. In some such implementations, the output signal is a voltage signal representative of an electrical voltage response of the tissues in the region of interest to the applied excitation signal. The detected voltage response signal is influenced by the different, and in some instances time-varying, electrical properties of the various tissues through which the injected excitation current signal is passed. In some implementations in which the bioimpedance sensor is operable to monitor blood pressure, heartrate or other cardiovascular characteristics, the detected voltage response signal is amplitude- and phase-modulated by the time-varying impedance (or inversely the admittance) of the underlying arteries, which fluctuates synchronously with the user's heartbeat as described above. To determine various biological characteristics, information in the detected voltage response signal is generally demodulated from the excitation carrier frequency component using various analog or digital signal processing circuits, which can include both passive and active components.

In some examples incorporating ultrasound sensors, measurements of arterial distension may involve directing ultrasonic waves into a limb towards an artery, for example, via one or more ultrasound transducers. Such ultrasound sensors also are configured to receive reflected waves that are based, at least in part, on the directed waves. The reflected waves may include scattered waves, specularly reflected waves, or both scattered waves and specularly reflected waves. The reflected waves provide information about the arterial walls, and thus the arterial distension.

In some implementations, regardless of the type of sensors utilized for the first arterial distension sensor 406 and the second arterial distension sensor 408, both the first arterial distension sensor 406 and the second arterial distension sensor 408 can be arranged, assembled or otherwise included within a single housing of a single monitoring device. As described above, the housing and other components of the monitoring device can be configured such that when the monitoring device is affixed or otherwise physically coupled to a subject, both the first arterial distension sensor 406 and the second arterial distension sensor 408 are in contact with or in close proximity to the skin of the user at first and second locations, respectively, separated by a distance ΔD, and in some implementations, along a stretch of the artery between which various arterial properties can be assumed to be relatively constant. In various implementations, the housing of the monitoring device is a wearable housing or is incorporated into or integrated with a wearable housing. In some specific implementations, the wearable housing includes (or is connected with) a physical coupling mechanism for removable non-invasive attachment to the user. The housing can be formed using any of a variety of suitable manufacturing processes, including injection molding and vacuum forming, among others. In addition, the housing can be made from any of a variety of suitable materials, including, but not limited to, plastic, metal, glass, rubber and ceramic, or combinations of these or other materials. In particular implementations, the housing and coupling mechanism enable full ambulatory use. In other words, some implementations of the wearable monitoring devices described herein are noninvasive, not physically-inhibiting and generally do not restrict the free uninhibited motion of a subject's arms or legs, enabling continuous or periodic monitoring of cardiovascular characteristics such as blood pressure even as the subject is mobile or otherwise engaged in a physical activity. As such, the monitoring device facilitates and enables long-term wearing and monitoring (for example, over days, weeks or a month or more without interruption) of one or more biological characteristics of interest to obtain a better picture of such characteristics over extended durations of time, and generally, a better picture of the user's health.

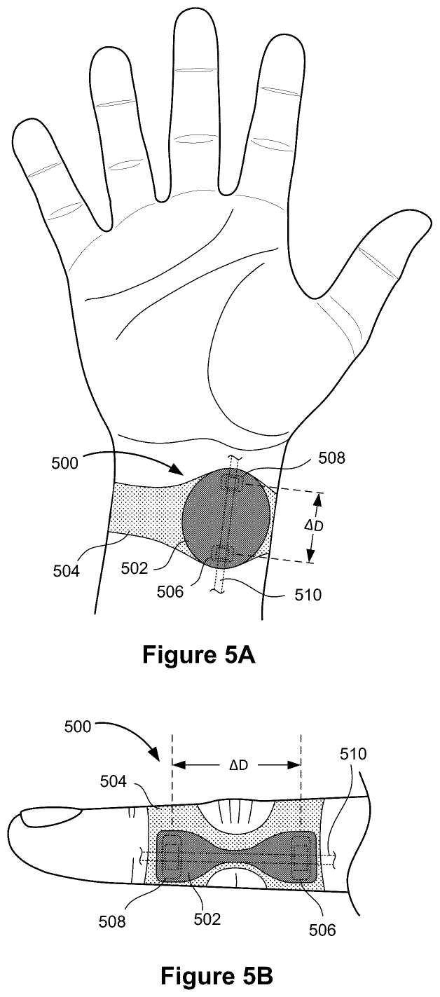

In some implementations, the monitoring device can be positioned around a wrist of a user with a strap or band, similar to a watch or fitness/activity tracker. FIG. 5A shows an example monitoring device 500 designed to be worn around a wrist according to some implementations. In the illustrated example, the monitoring device 500 includes a housing 502 integrally formed with, coupled with or otherwise integrated with a wristband 504. The first and the second arterial distension sensors 506 and 508 may, in some instances, each include an instance of the ultrasonic receiver system and a portion of the light source system that are described above. In this example, the monitoring device 500 is coupled around the wrist such that the first and the second arterial distension sensors 506 and 508 within the housing 502 are each positioned along a segment of the radial artery 510 (note that the sensors are generally hidden from view from the external or outer surface of the housing facing the subject while the monitoring device is coupled with the subject, but exposed on an inner surface of the housing to enable the sensors to obtain measurements through the subject's skin from the underlying artery). Also as shown, the first and the second arterial distension sensors 506 and 508 are separated by a fixed distance ΔD. In some other implementations, the monitoring device 500 can similarly be designed or adapted for positioning around a forearm, an upper arm, an ankle, a lower leg, an upper leg, or a finger (all of which are hereinafter referred to as “limbs”) using a strap or band.

FIG. 5B shows an example monitoring device 500 designed to be worn on a finger according to some implementations. The first and the second arterial distension sensors 506 and 508 may, in some instances, each include an instance of the ultrasonic receiver and a portion of the light source system that are described above.

In some other implementations, the monitoring devices disclosed herein can be positioned on a region of interest of the user without the use of a strap or band. For example, the first and the second arterial distension sensors 506 and 508 and other components of the monitoring device can be enclosed in a housing that is secured to the skin of a region of interest of the user using an adhesive or other suitable attachment mechanism (an example of a “patch” monitoring device).

FIG. 5C shows an example monitoring device 500 designed to reside on an earbud according to some implementations. According to this example, the monitoring device 500 is coupled to the housing of an earbud 520. The first and second arterial distension sensors 506 and 508 may, in some instances, each include an instance of the ultrasonic receiver and a portion of the light source system that are described above.

### Sensor Apparatus

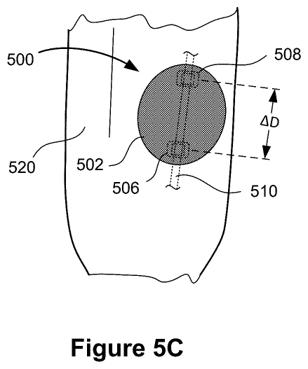

FIG. 6 is a block diagram that shows example components of an apparatus 600 according to some implementations. In some examples, the apparatus 600 may include an interface 601 and a force sensing system 602 and an object sensing system 604, e.g., an acoustic sensing system and/or a photoacoustic sensing system.

Some implementations of the apparatus 600 may include a control system 606, a communication interface system 608, a noise reduction system 610, or a combination thereof, each of which will be described in detail further below.

In some configurations, the apparatus 600 may be implemented with a device such as that listed elsewhere above, such as a sensor apparatus 700 (discussed below). In some implementations, components of the apparatus 600 may operate in concert with one another.

In some embodiments, the interface 601 may be or include a sensing portion or sensing area of a housing or chassis for the apparatus 600, or a platen of the apparatus 600. In some configurations, the interface 601 may be along the same central axis as the force sensing system 602 and/or the object sensing system 604 or a sensor associated therewith. In some configurations, the interface 601 may not be along the same central axis as the force sensing system 602 and/or the object sensing system 604 or a sensor associated therewith. Hence, the interface 601 may be optically and/or acoustically transparent, allowing for optical signals (e.g., light, IR) and acoustic signals (including, e.g., ultrasound waves) to be transmitted and received through the interface 601.

In some embodiments, the force sensing system 602 may be configured to perform force detection and measurement. In some examples, the force sensing system 602 may be or include a capacitive sensor. That is, the force sensing system 602 may include at least a force-sensing capacitor having a material whose capacitance changes when a force, pressure, or mechanical stress is applied thereto. In some implementations, such a sensor element may have compact dimensions that can be implemented with miniature (e.g., handheld) devices (e.g., up to 0.3 mm thick and up to 15 mm in diameter) while being sensitive enough to detect small pressures (e.g., down to 0.2 grams of force) with low error (e.g., under 1%).

In some examples, the force sensing system 602 may be or include a load cell, which may use resistors as a strain gauging transducer element to determine the amount of load transferred by mechanical force. A load cell may detect force by various other means, e.g., piezoresistivity, inductance, reluctance, magnetostrictive.

Example dimensions of sensor elements and strain gauges may be up to 0.3 mm thick and up to 15 mm in diameter, or even on the micron scale, e.g., 100 μm or 1000 μm across.

It may be useful to localize the detection of external pressure experienced by the target object (e.g., blood vessel). Having the aforementioned sensing area for force would not take into account force applied to the surrounding housing of the sensor apparatus. However, in some configurations, multiple force sensors, load cells, etc. of the force sensing system 602 may be implemented, which may ensure that the force is evenly distributed along the target object in the area of measurement.

Knowledge of the external pressure (counterpressure) is a key factor that may be determined using the force sensing system 602. Counterpressure experienced by the target object may be a function of the force measured by the force sensing system 602, and the surface area of the sensing area (e.g., force divided by area).

In some embodiments, the force sensing system 602 may be internal to the apparatus 600. That is, the force sensing system 602 may be disposed inside a housing or chassis of the apparatus 600. In such configurations, force applied to an outside surface of the apparatus 600 may be mechanically transferred to the force sensing system 602 inside the housing or chassis, e.g., via the interface 601. An example form factor of the housing or chassis of the apparatus 600 may be a probe, such as a handheld probe that can be applied against a body part of a user. Simplified depictions of the probe are shown in FIGS. 7A, 7B, 8A-8C, 9A and 9B. Another example form factor of the housing or chassis may be a ring or band, such as that shown in FIGS. 5A-5C, which may be worn by a user around a wrist, finger, toe, arm, leg, ankle, neck, waist, or another appendage, or another portion of the body. In an example implementation, the wearable device may have the form of a wristwatch and can be worn around the wrist. Hence, apparatus 600 may be constructed as various types of wearable and/or cuffless form factors that do not apply significant external pressure to the user. However, the embodiments described herein are not so limited. In certain cases, the components of the apparatus 600 may not all be worn.

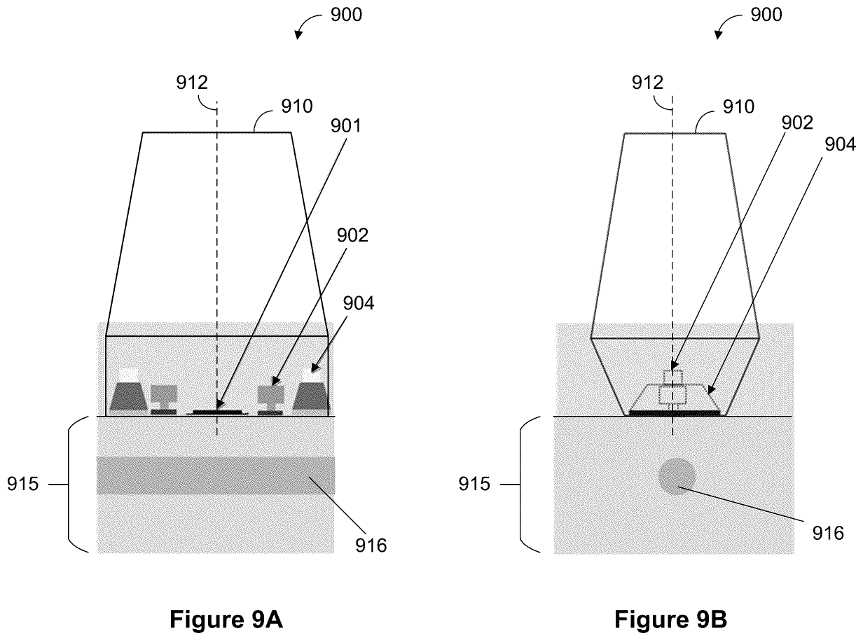

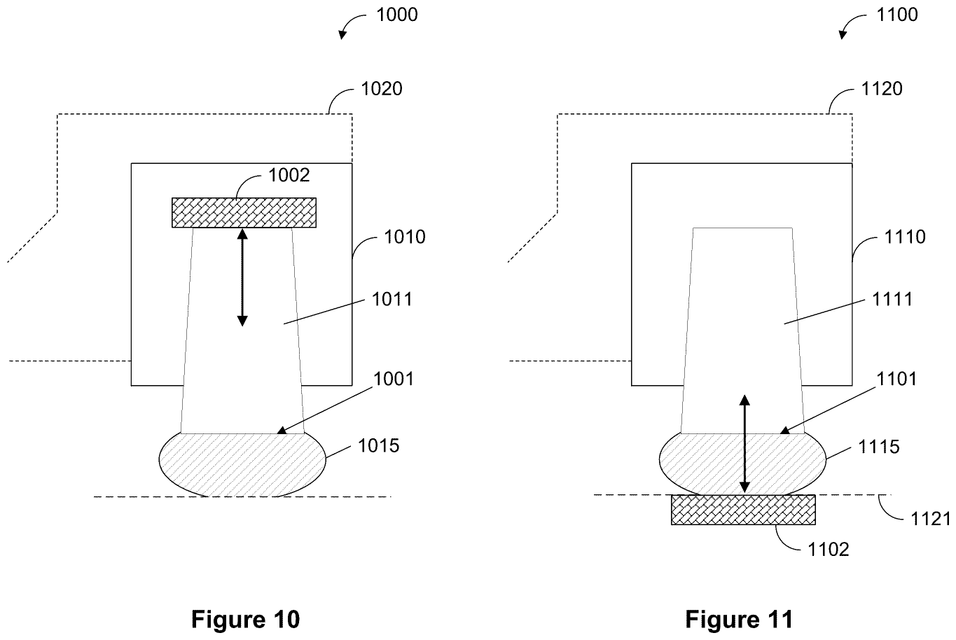

In some embodiments, the force sensing system 602 may be external to the apparatus 600. For example, the force sensing system 602 may be disposed at least partly outside a housing or chassis of the apparatus 600. For example, the apparatus 600 may be a benchtop device having a housing outside of a probe or other measuring device, with force sensing system 602 in the housing and against the measuring device. As another example, the force sensing system 602 may be configured for contact directly with the user's body part, or via another interface. Simplified depictions of the apparatus having an external force sensor are shown in FIGS. 10 and 11.

Hence, in some configurations, at least a portion of the interface 601 and at least a portion of the force sensing system 602 may be mechanically coupled to each other.

The object sensing system 604 may include one or more of various types of sensors. In some embodiments, at least an acoustic sensing system 604a may be included. Examples of acoustic sensing system 604a may include a photoacoustic (PAPG) sensor system, a pulse echo ultrasound (PeU) sensor system, and/or an acoustic (e.g., microphone) sensor system.

### Photoacoustic Sensor System

In some implementations, a photoacoustic sensor system may be included, which may include an interface, a light source system, a receiver system, and may be an example of the blood pressure monitoring device based on PAPG as shown in FIG. 1. In some implementations, the photoacoustic sensor system may also include a controller system or a controller, and/or share control functionality with control system 606.

Some disclosed PAPG sensors described herein (such as the aforementioned photoacoustic sensor system) may include a platen, a light source system, and an ultrasonic receiver system. According to some implementations, the light source system may include a light source configured to produce and direct light. In some implementations, the platen may include an anti-reflective layer, a mirror layer, or combinations thereof. According to some implementations, the platen may have an outer surface, or a layer on the outer surface, with an acoustic impedance that is configured to approximate the acoustic impedance of human skin. In some implementations, the platen may have a surface proximate the ultrasonic receiver system, or a layer on the surface proximate the ultrasonic receiver system, with an acoustic impedance that is configured to approximate the acoustic impedance of the ultrasonic receiver system.

Some disclosed PAPG sensors described herein (such as the aforementioned photoacoustic sensor system) may include an interface, a light source system and an ultrasonic receiver system. Some such devices may not include a rigid platen. According to some implementations, the interface may be a physical, flexible interface constructed of one or more of suitable materials having a desired property or properties (e.g., an acoustic property such as acoustic impedance, softness of the material). In some implementations, the interface may be a flexible interface that can contact a target object that may be proximate to or contact the interface. There may be salient differences between such an interface and a platen. In some implementations, the light source system may be configured to direct light using one or more optical waveguides (e.g., optical fibers) configured to direct light toward a target object. According to some implementations, the interface may have an outer surface, or a layer on the outer surface, with an acoustic impedance that is configured to approximate the acoustic impedance of human skin. Such outer surface may have a contact portion that is contactable by a user or a body part of the user (e.g., finger, wrist). In some examples, the optical waveguide(s) may be embedded in one or more acoustic matching layers that are configured to bring the light transmitted by the optical waveguide(s) very close to tissue. The outer surface and/or other parts of the interface may be compliant, pliable, flexible, or otherwise at least partially conforming to the shape and contours of the body part of the user. In some implementations, the interface may have a surface proximate the ultrasonic receiver system, or a layer on the surface proximate the ultrasonic receiver system, with an acoustic impedance that is configured to approximate the acoustic impedance of the ultrasonic receiver system.

In some embodiments, the light source system may, include one or more light-emitting diodes. In some implementations, the light source system may include one or more laser diodes. According to some implementations, the light source system may include one or more vertical-cavity surface-emitting lasers (VCSELs). In some implementations, the light source system may include one or more edge-emitting lasers. In some implementations, the light source system may include one or more neodymium-doped yttrium aluminum garnet (Nd:YAG) lasers.

Hence, the light source system may include, for example, a laser diode, a light-emitting diode (LED), or an array of either or both. The light source system may be configured to generate and emit optical signals. The light source system may, in some examples, be configured to transmit light in one or more wavelength ranges. In some examples, the light source system may be configured to transmit light in a wavelength range of 500 to 600 nanometers (nm). According to some examples, the light source system may be configured to transmit light in a wavelength range of 800 to 950 nm. According to some examples, the light source system may be configured to transmit light in infrared or near infrared (NIR) region of the electromagnetic spectrum (about 700 to 2500 nm). In view of factors such as skin reflectance, fluence, the absorption coefficients of blood and various tissues, and skin safety limits, one or both of these wavelength ranges may be suitable for various use cases. For example, the wavelength ranges of 500 nm to 600 nm and of 800 to 950 nm may both be suitable for obtaining photoacoustic responses from relatively smaller, shallower blood vessels, such as blood vessels having diameters of approximately 0.5 mm and depths in the range of 0.5 mm to 1.5 mm, such as may be found in a finger. The wavelength range of 800 to 950 nm, or about 700 to 900 nm, or about 600 to 1100 nm may, for example, be suitable for obtaining photoacoustic responses from relatively larger, deeper blood vessels, such as blood vessels having diameters of approximately 2.0 mm and depths in the range of 2 mm to 3 mm, such as may be found in an adult wrist. In some implementations, the light source system may be configured to switch wavelengths to capture acoustic information from different depths, e.g., based on signal(s) from a controller of the photoacoustic sensor system.

In some implementations, the light source system may be configured for emitting various wavelengths of light, which may be selectable to trigger acoustic wave emissions primarily from a particular type of material. That is, light sources may correspond to visible light, infrared light, or both. For example, because the hemoglobin in blood absorbs near-infrared light very strongly, in some implementations the light source system may be configured for emitting one or more wavelengths of light in the near-infrared range, in order to trigger acoustic wave emissions from hemoglobin. However, in some examples, the controller and/or control system (e.g., 606) may control the wavelength(s) of light emitted by the light source system to preferentially induce acoustic waves in blood vessels, other soft tissue, and/or bones. For example, an infrared (IR) light-emitting diode LED may be selected and a short pulse of IR light emitted to illuminate a portion of a target object and generate acoustic wave emissions that are then detected by the receiver system. In another example, an IR LED and a red LED or other color such as green, blue, white or ultraviolet (UV) may be selected and a short pulse of light emitted from each light source in turn with ultrasonic images obtained after light has been emitted from each light source. In other implementations, one or more light sources of different wavelengths may be fired in turn or simultaneously to generate acoustic emissions that may be detected by the ultrasonic receiver. Image data from the ultrasonic receiver that is obtained with light sources of different wavelengths and at different depths (e.g., varying range gate delays (RGDs)) into the target object may be combined to determine the location and type of material in the target object. Image contrast may occur as materials in the body generally absorb light at different wavelengths differently. As materials in the body absorb light at a specific wavelength, they may heat differentially and generate acoustic wave emissions with sufficiently short pulses of light having sufficient intensities. Depth contrast may be obtained with light of different wavelengths and/or intensities at each selected wavelength. That is, successive images may be obtained at a fixed RGD (which may correspond with a fixed depth into the target object) with varying light intensities and wavelengths to detect materials and their locations within a target object. For example, hemoglobin, blood glucose or blood oxygen within a blood vessel inside a target object such as a finger may be detected photoacoustically.

According to some implementations, the light source system may be configured for emitting a light pulse with a pulse width less than about 100 nanoseconds. In some implementations, the light pulse may have a pulse width between about 10 nanoseconds and about 500 nanoseconds or more. According to some examples, the light source system may be configured for emitting a plurality of light pulses at a pulse repetition frequency between 10 Hz and 100 kHz. Alternatively, or additionally, in some implementations the light source system may be configured for emitting a plurality of light pulses at a pulse repetition frequency between about 1 MHz and about 100 MHz. Alternatively, or additionally, in some implementations the light source system may be configured for emitting a plurality of light pulses at a pulse repetition frequency between about 10 Hz and about 1 MHz. In some examples, the pulse repetition frequency of the light pulses may correspond to an acoustic resonant frequency of the ultrasonic receiver and the substrate. For example, a set of four or more light pulses may be emitted from the light source system at a frequency that corresponds with the resonant frequency of a resonant acoustic cavity in the sensor stack, allowing a build-up of the received ultrasonic waves and a higher resultant signal strength. In some implementations, filtered light or light sources with specific wavelengths for detecting selected materials may be included with the light source system. In some implementations, the light source system may contain light sources such as red, green and blue LEDs of a display that may be augmented with light sources of other wavelengths (such as IR and/or UV) and with light sources of higher optical power. For example, high-power laser diodes or electronic flash units (e.g., an LED or xenon flash unit) with or without filters may be used for short-term illumination of the target object.

According to some examples, the light source system may also include one or more light-directing elements configured to direct light from the light source system towards the target object along the first axis. In some examples, the one or more light-directing elements may include at least one diffraction grating. Alternatively, or additionally, the one or more light-directing elements may include at least one lens.

In some example implementations, some or all of the one or more light sources may be disposed at or along an axis that is parallel to or angled relative to a central axis associated with the interface or platen. Optical signals may be emitted toward the target object (e.g., blood vessel), which may cause generation of ultrasonic waves by the target object. These ultrasonic waves may be detectable by one or more receiver elements.

In various configurations, the light source system may incorporate anti-reflection (AR) coating, a mirror, a light-blocking layer, a shield to minimize crosstalk, etc.

The light source system may include various types of drive circuitry, depending on the particular implementation. In some disclosed implementations, the light source system may include at least one multi-junction laser diode, which may produce less noise than single-junction laser diodes. In some examples, the light source system may include a drive circuit (also referred to herein as drive circuitry) configured to cause the light source system to emit pulses of light at pulse widths in a range from 3 nanoseconds to 1000 nanoseconds. According to some examples, the light source system may include a drive circuit configured to cause the light source system to emit pulses of light at pulse repetition frequencies in a range from 1 kilohertz to 100 kilohertz.

Various examples of a receiver system are disclosed herein, some of which may include an acoustic receiver system, an optical receiver system, or a combination thereof. In some implementations, the receiver system includes an acoustic receiver system (e.g., an ultrasonic receiver system) having the one or more receiver elements. In some examples, the receiver system may include a piezoelectric receiver layer, such as a layer of PVDF polymer or a layer of PVDF-TrFE copolymer. In some implementations, a single piezoelectric layer may serve as an ultrasonic receiver. In some implementations, other piezoelectric materials may be used in the piezoelectric layer, such as aluminum nitride (AlN) or lead zirconate titanate (PZT). The receiver system may, in some examples, include an array of ultrasonic transducer elements, such as an array of piezoelectric micromachined ultrasonic transducers (PMUTs), an array of capacitive micromachined ultrasonic transducers (CMUTs), etc. In some such examples, a piezoelectric receiver layer, PMUT elements in a single-layer array of PMUTs, or CMUT elements in a single-layer array of CMUTs, may be used as ultrasonic transmitters as well as ultrasonic receivers. According to some examples, the receiver system may be, or may include, an ultrasonic receiver array. In some examples, the photoacoustic sensor system may include one or more separate ultrasonic transmitter elements or one or more separate arrays of ultrasonic transmitter elements. In some examples, the ultrasonic transmitter(s) may include an ultrasonic plane-wave generator.

The acoustic signals (e.g., ultrasonic waves) emitted from acoustic transmitter system may cause or result in reflection of acoustic wave emissions at least in part from the object (e.g., finger). Characteristics of the reflected waves such as amplitudes may depend in part on the acoustic properties of the object and/or the platen. These reflected acoustic waves (e.g., ultrasonic waves) may be detectable by the acoustic receiver system.

In some implementations, at least portions of the photoacoustic sensor system (for example, the receiver system, the light source system, or both) may include one or more sound-absorbing layers, acoustic isolation material, light-absorbing material, light-reflecting material, or combinations thereof. In some examples, acoustic isolation material may reside between the light source system and at least a portion of the receiver system. In some examples, at least portions of the photoacoustic sensor system (for example, the receiver system, the light source system, or both) may include one or more electromagnetically shielded transmission wires. In some such examples, the one or more electromagnetically shielded transmission wires may be configured to reduce electromagnetic interference from the light source system that is received by the receiver system.

In the context of the present disclosure, a transmitter element and a receiver element may collectively or individually be referred to as a “sensing element,” an “acoustic sensing element,” a “sensor element,” or an “acoustic sensor element.” Such an element may also refer to a transceiver element or an acoustic transceiver element. In some instances, the foregoing terms may refer collectively to a transmitter element (e.g., of an acoustic transmitter system) and a receiver element (e.g., of an acoustic receiver system) that share the same piezoelectric layer.

### Pulse Echo Ultrasound (PeU) Sensor System

A PeU sensor system may implement a transducer configured to generate and send an acoustic signal (e.g., ultrasonic pulse) into a material (e.g., a body part), and a receiver configured to receive an echo—a reflected acoustic signal from the material. In some implementations, the transducer and the receiver may each be a piezoelectric transducer, such as the type described above.

The echo may be a portion of the acoustic signal that is reflected back from the material, e.g., from a surface or boundary of tissue. Another portion of the acoustic signal may continue passing through the material and reflect back from another boundary. In some scenarios, differences in acoustic impedance of tissues may cause some ultrasound pulses to be reflected and some to be transmitted.

In some approaches, the PeU sensor system may transmit the acoustic signal during a transmit time and listen for echoes during a receive time. Received echoes may be used to determine tissue boundaries and distances. Ultrasound pulses (and transmit parameters such as pulse duration (time taken for a pulse to be emitted from the PeU sensor system) and spatial pulse length (distance that the pulse travels through space)) and echoes can be used to determine distances to a target object (e.g., blood vessel) and/or image tissue anatomy. For example, tissue boundaries can be plotted based on receive time of echoes. These determinations can allow the PeU sensor system to, e.g., detect changes in arterial diameter caused by pulsatile blood flow. Such physical characteristics may be useful data for determining physiological parameters of a user, such as blood pressure.

### Acoustic Sensor System

In some implementations, the acoustic sensor system may include an acoustic transmitter system and an acoustic receiver system. The acoustic transmitter system may be configured to generate and emit acoustic signals, e.g., toward a target object, such as a finger or other object. Acoustic signals may include one or more acoustic waves. In some implementations, the acoustic transmitter system may include one or more ultrasonic transmitters or transmitter elements configured to generate, emit, and/or direct ultrasonic waves. The one or more ultrasonic transmitters may be one or more ultrasonic transducers. In some implementations, ultrasonic waves may be generated in a selected portion of multiple ultrasound transmitter elements (e.g., in an array). In some configurations, the one or more ultrasonic transmitter elements may be arranged in an array of ultrasonic transducer elements, such as an array of PMUTs and/or an array of CMUTs. In some examples, the ultrasonic transmitter(s) may include an ultrasonic plane-wave generator.

In some examples, the acoustic transmitter system may include one or more microphone elements. Each microphone may be a MEMS microphone having components enabling acoustic transmission and reception, such as an inlet port, a cavity, and/or a membrane or mesh to facilitate detection and receipt of acoustic signals (e.g., sound waves) from a body part of the user (such as the finger 115 shown in FIG. 1). In some cases, microphones may be coupled using a gasket creating cavities to amplify sound. The membrane or mesh may be a thin layer that separates the cavity and inlet port from the skin, and protects the microphone components from dust, sweat, etc.

In an example scenario, a blood vessel may generate acoustic signals. Pressure waves may be created by blood pressure within the blood vessel and/or variations in blood pressure and associated movements (e.g., distension or contraction) of the blood vessel. The pressure waves can produce sound waves in the tissue of the body part, which may be detected by the microphone via the membrane or mesh, the cavity, and then the inlet port.

Various configurations and designs of a microphone and its components may be envisioned by those having ordinary skill in the relevant arts. For instance, the shape of the cavity may be narrower or wider depending on the configuration. Example dimensions of a microphone may be 3 mm by 3 mm in size. A microphone may provide digital or analog signals measuring sound pressure level (SPL) at the inlet port. Heart rate waveforms can have relatively low frequencies (e.g., under 20 Hz). To detect sound waves from the blood vessel, sensitivity of the microphone may be very high, e.g., able to detect low frequencies at 1-10 Hz. Since some microphones can detect dynamic signals, heart rate signals detected by microphones can also appear as “high pass” signals of true heart rate waveforms filtering very low frequencies.

Depending on the configuration, a microphone may be based on capacitive, piezoelectric, and/or other sensing modalities. Examples may include a piezoelectric MEMS microphone or a capacitive MEMS microphone.

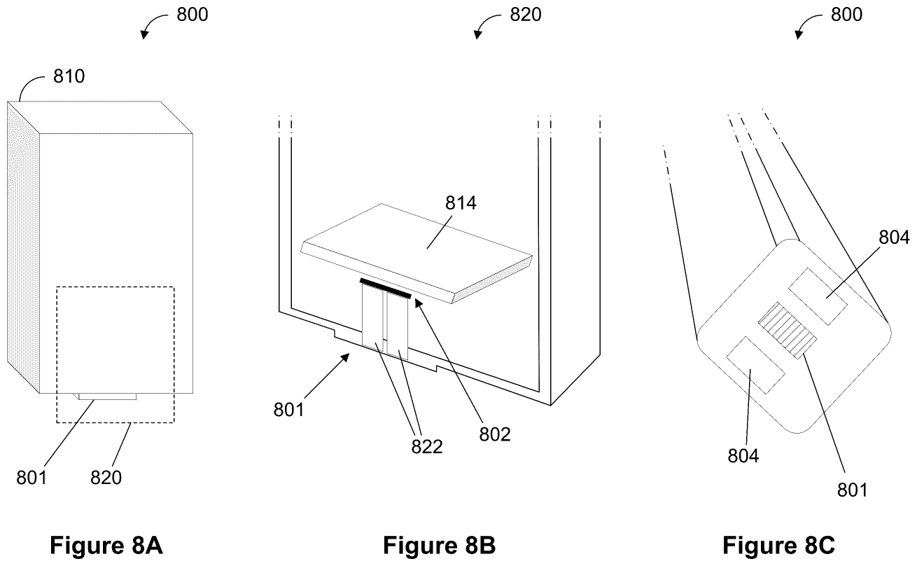

In some embodiments, at least one additional microphone may be disposed along the blood vessel at a distance from another microphone. This may allow measurements to be taken with a time delay, which is useful for estimating parameters such as pulse wave velocity. Example configurations of multiple microphones are depicted in FIGS. 7A and 9A.

In certain embodiments, an acoustic sensor system may include filters such as a low-pass filter configured to remove noise sources in higher frequencies (e.g., over 20 Hz from noise or motion-related artifacts) and support low-frequency acoustic signals. In some cases, a high-pass filter may be used to filter out lower frequencies of acoustic signals instead (e.g., under 1 Hz from motion or other sources such as breathing). In addition, digital signal processing (e.g., in a built-in ASIC) may be used by an acoustic sensor system to improve the quality of acoustic signals.

In some implementations, the acoustic transmitter system may include one or more acoustic waveguides or ultrasonic waveguides (or other sound-directing elements) constructed to propagate and direct acoustic or ultrasonic waves toward a target location that does not have direct line of sight from at least a portion of one or more acoustic transmitter elements. Such waveguides may be useful in certain devices, e.g., foldable displays, or chasses that may optimize the locations of the acoustic transmitter system and the acoustic receiver system by placing them out of direct line of sight.

In some implementations, the acoustic receiver system may be an ultrasonic receiver system having one or more ultrasonic receiver elements of the types discussed above.

In implementations that include an acoustic receiver system, the acoustic receiver system (e.g., an ultrasonic receiver system having one or more ultrasonic receiver elements) and the acoustic transmitter system (e.g., an ultrasonic transmitter system having one or more ultrasonic transmitter elements) may be combined in an acoustic transceiver system (e.g., an ultrasonic transceiver system).

Accordingly, embodiments of apparatus 600 may be configured to operate as ultrasound sensors that are configured to receive reflected acoustic signals such as ultrasonic waves. Reflected ultrasonic waves may include scattered waves, specularly reflected waves, or both scattered waves and specularly reflected waves. The reflected waves can provide acoustic data, including information about the object, e.g., a finger's ridges and valleys and their shapes and patterns.

### Optical Sensing Systems

In some embodiments, at least an optical sensing system 604b may be included with the object sensing system 604. Examples of optical sensing system 604b may include a PPG sensor system and/or an IR sensor system.

In some implementations, a PPG sensor system may include at least an optical sensor system such as a PPG-based device configured to operate according to the principles described with respect to FIG. 2.

In some implementations, an IR sensor system may include an IR LED configured to produce infrared light of differing wavelengths within the infrared band (about 780 nm to 1 mm), and an IR photodiode that is sensitive to IR light. Similar to PPG, the IR sensor system may be used to detect volumetric changes in the blood, e.g., by measuring the intensity of the reflected IR light. The amount of IR light that passes through the skin depends on the amount of hemoglobin in the blood, which changes as blood volume increases.

### RF Sensing Systems

In some embodiments, at least an RF sensing system 604c may be included with the object sensing system 604. In some implementations, the RF sensing system 604c may perform techniques to detect changes in electrical impedance caused by pulsatile blood flow. For example, the RF sensing system 604c may be configured to perform electrical impedance spectroscopy (EIS) or electrical impedance tomography (EIT), or may include an electromagnetic coupling sensor to detect electrical impedance due to blood flow. EIS may be used to estimate changes in the diameter of the radial artery caused by blood flow. EIS can be used to measure impedance over time, which may yield data (e.g., heart rate waveforms) that reflect changes due to blood flow.

Such bioimpedance analysis can provide characteristics of a target object such as PTT and heart rate, which may be key to deriving other physiological parameters. As such, RF sensing system 604c may be used for determining and predicting arterial (e.g., blood pressure) measurements such as blood pressure.

Accordingly, various types of sensing systems as described above can be used to measure various physical and physiological characteristics, which will be useful in performing techniques in conjunction with external counterpressure sensing and calibration to estimate user parameters such as blood pressure as will be discussed below.

In some implementations, a control system 606 may include one or more controllers or processors, or a drive circuit or various types of drive circuitry, configured to control the one or more ultrasonic transmitter elements via one or more instructions to the acoustic transmitter system. For example, ultrasonic waves may be generated in pulses (e.g., at least partly repeating or other patterns) or according to other timing instructions. Although “ultrasound” may typically apply to acoustic energy with a frequency above human hearing, or 20 kilohertz (kHz), ultrasound frequencies used may exceed well over this lower limit. In some implementations, the control system 606 may cause ultrasonic waves from the acoustic transmitter system to be generated and emitted at a frequency that is between about 12 megahertz (MHz) to 50 MHz, which may result in sufficient resolution for, e.g., imaging, e.g., up to 1000 dots per inch (dpi). Other suitable frequencies may be used for the acoustic waves in other implementations.

Control system 606 may be electrically and/or communicatively coupled to the apparatus 600. In some configurations, the control system 606 may be part of the apparatus 600. In some configurations, the control system 606 may be part of a device having the apparatus 600. In some configurations, the control system 606 may be external to the apparatus 600 or the device having the apparatus 600, for example but not limited to, on a server (cloud), remote storage, or another device other than the device having the apparatus 600. In some configurations, the one or more controllers or processors of the control system 606 may be distributed across two or more devices including external apparatus.

In some implementations, the control system 606 may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The control system 606 also may include (and/or be configured for communication with) one or more memory devices, such as one or more random access memory (RAM) devices, read-only memory (ROM) devices, etc. Accordingly, the apparatus 600 may have a memory system that includes one or more memory devices, though the memory system is not shown in FIG. 6. The control system 606 may be configured for receiving and processing data from the receiver system, e.g., as described elsewhere herein. In some implementations, functionality of the control system 606 may be partitioned between one or more controllers or processors, such as a dedicated sensor controller and an applications processor of a mobile device. The control system 606 may be configured to perform or cause performance of certain operations using one or more of the above components.

If the apparatus 600 includes an ultrasonic transmitter, such as in the acoustic transmitter system, the control system 606 may be configured for controlling the ultrasonic transmitter. In some embodiments, a control system 606 may cause the acoustic transmitter system to generate and emit acoustic waves. In some implementations, the control system 606 may cause the acoustic transmitter system to generate and emit acoustic waves in response to a detection of an object (e.g., a finger). In some cases, the object may be detected based at least on a force applied to the apparatus 600. In some examples, a resistive sensor or touchscreen may allow detection of sufficient force applied to the apparatus 600.

In some cases, the object may be detected based at least on light occlusion. In such cases, a light sensor may also be included with the apparatus 600 so that an amount of light or its absence (e.g., relative to a threshold) can be determined, e.g., by control system 606, at or near the apparatus 600.

In some cases, the object may be detected based at least on a capacitive shift or response. For example, a capacitive sensor or touchscreen may allow determination of a capacitive response based on the natural conductivity of the object such as a finger that is making contact with the interface 601 of the apparatus 600.

In some implementations, a combination of one or more detection methods described above may be used to detect the object. For instance, detection of the object may require, in some configurations, sufficient force and sufficient capacitive response. In another example, detection of the object may require sufficient force, sufficient capacitive response, and sufficient absence of light. In some cases, said sufficient force may be detected using the force sensing system 602.

In some configurations, a delay may be placed between the detection of the object and the emission of the acoustic waves, where the length of the delay may be 100 milliseconds, 500 milliseconds, etc. Not causing emission of acoustic waves immediately may allow time for the object to stabilize against the apparatus 600 before obtaining measurements. Force or occlusion may occur even if the finger is not pressed onto the apparatus 600 completely.

In further examples, the control system 606 may be communicatively coupled to the receiver system. The receiver system may be configured to detect acoustic signals from the target object. The control system 606 may be configured to select at least one of a plurality of receiver elements of the receiver system. Such selected receiver element(s) may correspond to the best signals from multiple receiver elements. In some embodiments, the selection of the at least one receiver element may be based on information regarding detected acoustic signals (e.g., arterial signals or vein signals) from the plurality of receivers. For example, signal quality or signal strength (based, e.g., on signal-to-noise ratio (SNR)) of some signals may be relatively higher than some others or above a prescribed threshold or percentile, which may indicate the best signals. In some embodiments, control system 606 may be configured to receive the acoustic data (e.g., from acoustic receiver system) and/or generate images (e.g., three-dimensional images) representative of the object. In some implementations, the control system 606 may also be configured to, based on the information regarding detected acoustic signals, determine or estimate at least one characteristic of the blood vessels such as PWV (indicative of arterial stiffness), arterial dimensions, or others or combinations thereof.

Some implementations of the apparatus 600 may include a communication interface system 608. In some examples, the communication interface system 608 may include a wireless interface system. In some implementations, the communication interface system 608 may include a user interface system, one or more network interfaces, one or more communication interfaces between the control system 606 and a memory system, and/or one or more interfaces between the control system 606 and one or more external device interfaces (such as ports or applications processors), or combinations thereof. According to some examples in which the communication interface system 608 is present and includes a user interface system, the user interface system may include a microphone system (including, e.g., one or more microphones), a loudspeaker system, a haptic feedback system, a voice command system, one or more displays, or combinations thereof. According to some examples, the communication interface system 608 may include a touch sensor system, a gesture sensor system, or a combination thereof. The touch sensor system (if present) may be, or may include, a resistive touch sensor system, a surface capacitive touch sensor system, a projected capacitive touch sensor system, a surface acoustic wave touch sensor system, an infrared touch sensor system, any other suitable type of touch sensor system, or combinations thereof.

According to some examples, the apparatus 600 may include a noise reduction system 610. In some implementations, the noise reduction system 610 may include one or more sound-absorbing layers, acoustic isolation material, or combinations thereof. In some examples, the noise reduction system 610 may include acoustic isolation material, which may reside between at least a portion of the acoustic transmitter system and at least a portion of the acoustic receiver system, e.g., between ultrasonic transmitter elements and ultrasonic receiver elements. In some examples, the noise reduction system 610 may include one or more electromagnetically shielded transmission wires. In some such examples, the one or more electromagnetically shielded transmission wires may be configured to reduce electromagnetic interference from circuitry of the acoustic transmitter system, circuitry of the acoustic receiver system, or combinations thereof, that is received by the acoustic receiver system.

### Internal Force Sensors

FIG. 7A illustrates a cross-sectional view of an example configuration of a sensor apparatus 700 having an internal force sensor, according to some disclosed embodiments. The sensor apparatus 700 may be an example of the apparatus 600. In some implementations, the sensor apparatus 700 may include a housing 710 within which a force sensor 702 is contained. The force sensor 702 may be an example of the force sensing system 602, and may be a capacitive force sensor, a load cell, etc. as discussed above.

In some implementations, the sensor apparatus 700 and the housing 710 may be implemented as a handheld device with a sensing area 701 of a probe. In some configurations, the sensor apparatus 700 and the housing 710 may be implemented as a wearable device (wristband, ring, etc.). In some implementations, the force sensor 702 may be disposed and aligned substantially along a same central axis 712 as a sensing area 701 of the sensor apparatus 700. External pressure (counterpressure) applied by the sensor apparatus 700 (e.g., to user tissue 715 in a direction substantially orthogonal to the surface of the sensing area 701) may be measured by the force sensor 702. The measured force from the counterpressure may be advantageously used in estimating a physiological parameter of a user, such as blood pressure.

In addition, one or more sensors 704, which may be of one or various modalities (e.g., photoacoustic, PeU, acoustic, PPG, IR, and/or RF), may be an example of or part of the object sensing system 604. Thus, as discussed above, the one or more sensors 704 may be configured to transmit and receive signals (e.g., photoacoustic, ultrasonic, optical, and/or RF) through the sensing area 701 during operation and obtain measurements. During operation, the sensor apparatus 700 may be pressed against the user tissue 715, for instance, and cause a blood vessel 716 therein to be distorted. The applied counterpressure may change the diameter, distension, and/or cross-sectional area of the blood vessel 716.

In some implementations, the one or more sensors 704, while being part of the sensor apparatus 700, may not be co-located with the force sensor 702. Instead, the one or more sensors 704 may be adjacent to the force sensor 702 as shown, or disposed elsewhere.

FIG. 7B illustrates another cross-sectional view of an example configuration of a sensor apparatus 700 having an internal force sensor, according to some disclosed embodiments. The view of FIG. 7B is rotated 90 degrees with respect to the view of FIG. 7A. It can be seen that the force sensor 702 is adjacent to the one or more sensors 704 along a horizontal axis (not shown) that is parallel to the blood vessel 716. The force sensor 702 and the one or more sensors 704 is thus capable of obtaining respective measurements of counterpressure force and physical characteristics of the blood vessel 716.

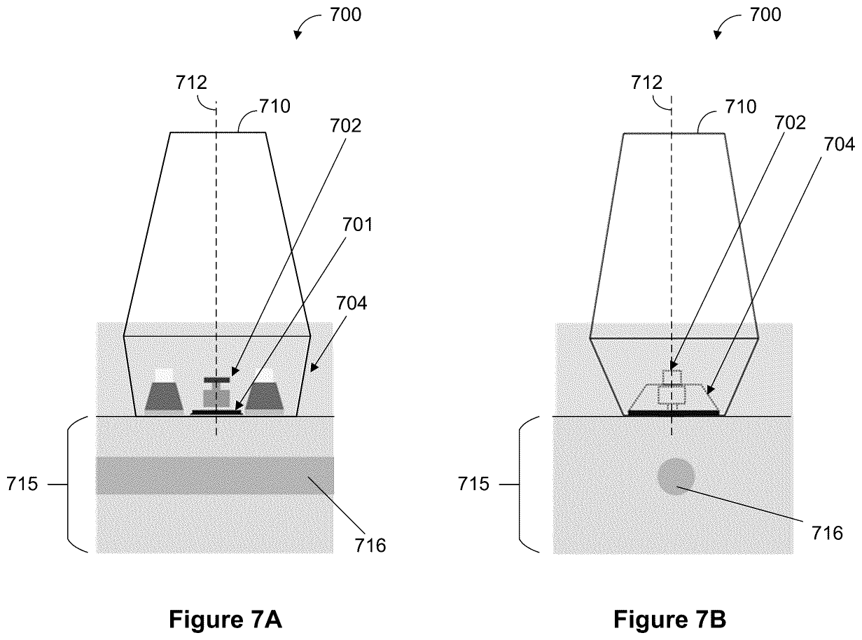

FIG. 8A illustrates a simplified representation of the example configuration of a sensor apparatus 800 having an internal force sensor, according to some disclosed embodiments. In some examples, the sensor apparatus 800 may be an example of the sensor apparatus 700 of FIGS. 7A and 7B, and may include a housing 810 with a probe sensing area 801 for interfacing with a user's tissue, and a force sensor 802 (shown in FIG. 8B) disposed inside the housing 810.

FIG. 8B illustrates a simplified representation of an internal view of a portion 820 of the sensor apparatus 800. Components inside the sensor apparatus 800 may include the force sensor 802 disposed inside the housing 810 and abutting one or more actuators 822 that can mechanical transfer external force (e.g., that applied to the sensing area 801). It may be noticed that, in some implementations, the force sensor 802 may have a compact dimensions having a particularly thin structure, which may be advantageous for implementing inside a handheld device or other compact form factor of the sensor apparatus 800 (a wearable device such as wristband, ring, etc.).

In various implementations, the sensor apparatus 800 may include various other components. For instance, a circuit board 814 and/or other electronic components may be included so as to, e.g., process and/or send detected force measurements to a controller system or processor(s).

FIG. 8C illustrates a simplified representation of another view of sensor apparatus 800 having an internal force sensor. The illustrated view may be, for instance, a bottom portion of the sensor apparatus 800, which reveals the sensing area 801 and sensing areas through which one or more sensors 804 may transmit and receive signals of the type noted with respect to the one or more sensors 704 or the object sensing system 604.

In some examples, the arrangement of the sensing area 801 and the one or more sensors 804 may correspond to the arrangement of the force sensor 702 and the one or more sensors 704 disposed adjacent to one another, as shown in FIG. 7A. Hence, externally applied force by the sensor apparatus 800 may be detected through the sensing area 801, and physiological measurements may be made through sensing areas of the one or more sensors 804.

FIG. 9A illustrates a cross-sectional view of another example configuration of a sensor apparatus 900 having an internal force sensor, according to some disclosed embodiments. Similar to the sensor apparatus 700, the sensor apparatus 900 may be an example of the apparatus 600, and may include a housing 910, a force sensor 902, and one or more sensors 904. The housing 910 may be an example of the housing 710. The one or more sensors 904 may be an example of the one or more sensors 704, and may be of one or various modalities as discussed above (e.g., photoacoustic, PeU, acoustic, PPG, IR, and/or RF). In some implementations, the sensor apparatus 900 and the housing 910 may be implemented as a handheld device with a sensing area 901 of a probe. In some configurations, the sensor apparatus 900 and the housing 910 may be implemented as a wearable device (wristband, ring, etc.).

The force sensor 902 may be an example of the force sensing system 602, and may be a capacitive force sensor, a load cell, etc. as discussed above. In some implementations, there may be multiple force sensors 902. However, in the illustrated example configuration of a sensor apparatus 900, the force sensor(s) 902 may be disposed outside a central axis 912 of the sensing area 901. As shown, the force sensor(s) 902 may be adjacent to the sensing area 901. Each the one or more sensors 904 may in turn be disposed adjacent to a force sensor 902 as shown, although other arrangements may be used. In some configurations, these components, including the sensing area 901 of the probe, the force sensor(s) 902, the one or more sensors 904, may be within housing 910. External pressure (counterpressure) applied by the sensor apparatus 900 (e.g., to user tissue 915 in a direction substantially orthogonal to the surface of the sensing area 901) may be measured by the force sensor(s) 902 proximate to the sensing area 901. The measured force from the counterpressure may be advantageously used in estimating a physiological parameter of a user, such as blood pressure.

FIG. 9B illustrates another cross-sectional view of the another example configuration of a sensor apparatus 900 having an internal force sensor, according to some disclosed embodiments. The view of FIG. 9B is rotated 90 degrees with respect to the view of FIG. 9A. It can be seen that the force sensor(s) 902 are adjacent to the one or more sensors 904 along a horizontal axis (not shown) that is parallel to the blood vessel 916. The force sensor(s) 902 and the one or more sensors 904 is thus capable of obtaining respective measurements of counterpressure force and physical characteristics of the blood vessel 916.

### External Force Sensors

FIG. 10 illustrates a cross-sectional view of an example configuration of a sensor apparatus 1000 having an external force sensor 1002, according to some disclosed embodiments. The sensor apparatus 1000 may be an example of the apparatus 600. In some implementations, the sensor apparatus 1000 may (or may not) include a housing 1010. The housing 1010 may be configured to contain and/or stabilize various components, such as a force sensor 1002, a sensing device 1011 having one or more sensors (such as object sensing system 604), and/or internal components such as circuit board(s), a controller system or processor(s), wiring and other connections, and/or other electronics (not shown). The force sensor 1002 may be an example of the force sensing system 602, and may be a capacitive force sensor, a load cell, etc. as discussed above. These various components may be further coupled with external equipment 1020, which may in various configurations include further housing, wiring, structure, scaffolding, display, etc.

In some implementations, the sensing device 1011 may include a probe having a sensing area 1001. In some implementations, the sensing device 1011 and/or an associated physical component be configured to transmit mechanical force to the force sensor 1002. In some cases, the force sensor 1002 may be a separate and discrete component disposed outside of the sensing device 1011. In some cases, the force sensor 1002 may be disposed outside of the housing 1010 (not depicted).

The mechanical force may be created by, e.g., a counterpressure created during operation of the sensor apparatus 1000 involving some applied pressure to a user's body part 1015 (e.g., wrist, arm). The force sensor 1002 may thereby be configured to determine an external pressure applied to the body part 1015. As discussed elsewhere herein, knowledge of the external pressure may be useful for estimating a physiological parameter such as blood pressure, which will be discussed later.

Since the force sensor 1002 may be placed outside of the sensing device 1011, the force sensor 1002 need not have small dimensions and may be of a type that can be more easily implemented and obtain more accurate force measurements. As an example, the sensor apparatus 1000 having an external force sensor 1002 may be in a benchtop configuration that is not in a compact form factor such as a handheld device or wearable device.

With sensor apparatus 1000 having an external force sensor 1002, the force applied to the force sensor 1002 may advantageously be uniform and consistent since the area of contact between the force sensor 1002 and the sensing device 1011 may be defined and the same each time mechanical force occurs from contacting the body part 1015. Moreover, with a force sensor external to a probe or other type of sensing apparatus, the same fixturing and force sensor could be used while allowing for modifications to the sensing apparatus or parts thereof, such as the sensor, transducer, etc.

In contrast, it may be more difficult with a sensor apparatus with an internal force sensor to determine how much pressure is distributed to an outside enclosure (e.g., housing) versus the sensing area (e.g., 801).

FIG. 11 illustrates a cross-sectional view of another example configuration of a sensor apparatus 1100 having an external force sensor 1102, according to some disclosed embodiments. The sensor apparatus 1100 may be an example of the apparatus 600. Similar to the sensor apparatus 1000, the sensor apparatus 1100 may (or may not) include a housing 1110. The housing 1110 may be configured to contain and/or stabilize various components, such as a sensing device 1111 having one or more sensors (such as object sensing system 604) and/or internal components such as circuit board(s), a controller system or processor(s), wiring and other connections, and/or other electronics (not shown). The force sensor 1102 may be an example of the force sensing system 602, and may be a capacitive force sensor, a load cell, etc. as discussed above. These various components may be further coupled with external equipment 1020, which may in various configurations include further housing, wiring, structure, scaffolding, display, etc.

In some implementations, the force sensor 1102 may be disposed separately from the housing 1110 and/or the sensing device 1111. In some configurations, the force sensor 1102 may be communicatively coupled (wired or wireless) with one or more of the aforementioned components. Hence, counterpressure created during operation of the sensor apparatus 1100 may be measured from the force sensor 1102 in a different location from force sensor 1002. In some configurations, force sensor 1102 may abut a platen 1121 or other surface associated with the sensor apparatus 1100. In some cases, a body part 1115 may directly contact force sensor 1102 a surface thereof during operation. The force sensor 1102 may thereby be configured to determine an external pressure applied to the body part 1115. As discussed elsewhere herein, knowledge of the external pressure may be useful for estimating a physiological parameter such as blood pressure, which will be discussed later.

As will now be discussed, the various aforementioned configurations of sensing apparatus having internal or external force sensors and biometric sensor systems may be used to estimate physiological parameters (e.g., blood pressure) based on physical characteristics of a target object (e.g., blood vessel).

### Calibration of Counterpressure

In some approaches, external pressure may be used to estimate blood pressure. Consider Equations 1 and 2 below.

$$α = \frac{{A}_{dia .} ln ( {P}_{sys .} / {P}_{dia .} )}{{A}_{sys .} - {A}_{dia .}} \qquad ( Eqn . 1 )$$

$$P ( t ) = {P}_{0} exp [ α ( \frac{A ( t )}{{A}_{0}} - 1 ) ] \qquad ( Eqn . 2 )$$

A_{sys.} represents a cross-sectional area of a blood vessel during systole. A_{dia.} represents a cross-sectional area of the blood vessel during diastole. P_{sys.} represents systolic blood pressure. P_{dia.} represents diastolic blood pressure. P_{sys.} and P_{dia.} may be obtained using sphygmomanometer or other conventional blood pressure gauge. A_{sys.} and A_{dia.} may be determined from signal measurements, e.g., using PAPG, PeU, PPG, acoustics, IR, and/or RF as described above. A calibration factor, which is herein referred to as alpha (α), can be derived from these four measurements according to Eqn. 1. A₀ represents a reference cross-sectional area of the blood vessel, e.g., during diastole. P₀ represents a reference pressure, e.g., an external counterpressure applied to the blood vessel. In some approaches, P₀ may be determined and known using a force sensor (e.g., force sensing system 602) implemented with a sensing apparatus as discussed above. In some approaches, during calibration, A₀ may be a known value equivalent to A_{dia.}, and P₀ may be a known value equivalent to P_{dia.}. A(t) represents a cross-sectional area of the blood vessel at a given time. P(t) represents blood pressure at a given time, which can be determined according to Eqn. 2 based at least on a obtained using Eqn. 1 and known reference counterpressure (P₀) and cross-sectional area (A₀). In some cases, P(t) may be a waveform representing heart rate cycles, and may include a set of blood pressure values over time, including during systole and diastole.

Alpha is a property of the blood vessel, so it may be dependent on a given user's unique physiology. In some cases, alpha may change over time. For these reasons, an initial calibration may be useful to determine a relationship between alpha and applied counterpressure.

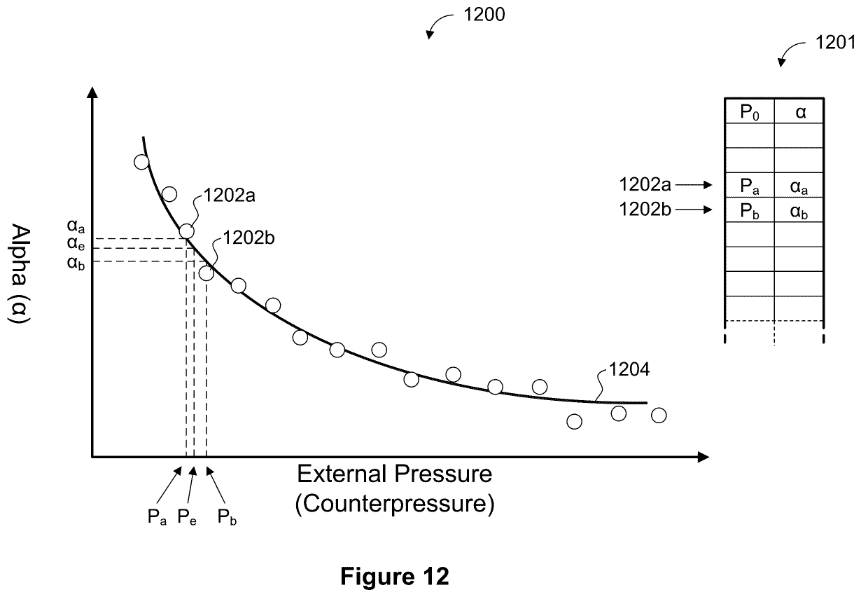

FIG. 12 is an example graph 1200 of external pressure as a function of calibration factor (alpha or α), representative of calibration data useful with some disclosed embodiments. The example graph 1200 may represent a relationship between external pressure (P₀) and a calibration factor (α) useful for determining blood pressure. In some approaches, the example graph 1200 may be generated based on manual or empirical measurements of arterial cross-sectional area, systolic blood pressure, and diastolic blood pressure at different external pressures to obtain varying values of alpha based on Eqn. 1. Predetermined external pressures may be applied using conventional means, such as a sphygmomanometer or other blood pressure gauge, which may involve an inflatable cuff for calibration purposes.

Values of applied external pressures and resulting values of alpha may be stored in a look-up table 1201, comma-separated values (CSV) data structure, or other types of data structure known to those having ordinary skill in the relevant arts. From these initial calibration values, example graph 1200 including at least data points 1202a, 1202b each corresponding to P₀ and α may be produced and used in some implementations.

In some cases, a curve 1204 may also be generated based on the data points (including 1202a, 1202b). In some approaches, curve 1204 may be fitted (e.g., polynomial, exponential, linear) based on the data points. The curve 1204 may be used as a look-up curve in some instances so as to be able to use extrapolated values. For example, according to fitted curve 1204, an external pressure Pₐ correlates to calibration factor αₐ, and external pressure P_{b} correlates to calibration factor α_{b}. If an external pressure Pₑ is applied, an extrapolated calibration factor αₑ could be estimated along the fitted curve 1204 advantageously providing a continuous or at least more granular look-up curve as compared to a more discrete look-up table such as look-up table 1201.

In various cases, some or all of the above types of information may be used as calibration data specific to a particular user when estimating blood pressure. In some approaches, the calibration data can be obtained by: (1) Using an external cuff to accurately measure P_{sys.} and P_{dia.}. (2) Measuring A_{sys.} and A_{dia.} over one or more cycles at one value of an external pressure (applied at a known value by, e.g., an external cuff). (3) Varying the value of the external pressure and repeating (2). (4) Repeating (2) and (3) one or more times (for a total of, e.g., 2-10 data points).

In some cases, the data points may be averaged or weighted averaged or used to determine certainty information for the data points (e.g., error bars). In some cases, this process may be performed by ramping up and down the external pressure slowly while collecting the A_{sys.} and A_{dia.} data and determining the calibration factor alpha. In some cases, alpha and external pressure can be correlated to each other and/or plotted for the user (e.g., as shown in example graph 1200) to obtain user-specific calibration data.

In some approaches, a machine learning (ML) model may be trained, e.g., using regression (e.g., polynomial, exponential, linear) and loss optimization, to obtain values of alpha given applied external pressures. In some cases, the relationship between alpha and applied external pressures may be output based on one or more sets of empirical measurements that may result in, e.g., look-up table 1201 or example graph 1200 as described above. In some cases, the relationship may be further finetuned based on previous one or more sets of empirical measurements. That is, previous output(s) for a user may be used as training data.

In some cases, the relationship may be determined based on empirical measurements from the user as well as empirical measurements or calibrated data from one or more other users as training data.

In some cases, the relationship may be determined based on characteristics of the user. For example, a ML model may determine a further relationship between external pressures, alpha, and other type(s) of information such as a physical characteristic of the blood vessel (e.g., distension or PWV, which may be determined using sensing techniques such as PAPG, PeU, PPG, acoustics, IR, and/or RF), or another characteristic of the user unrelated to alpha (age, location, gender, cholesterol, etc.).

A curve 1204 may be fitted on data points generated using the above ML-based techniques or from empirical observations as discussed above. In some cases, the curve 1204 may be discarded if its R² coefficient is below a threshold.

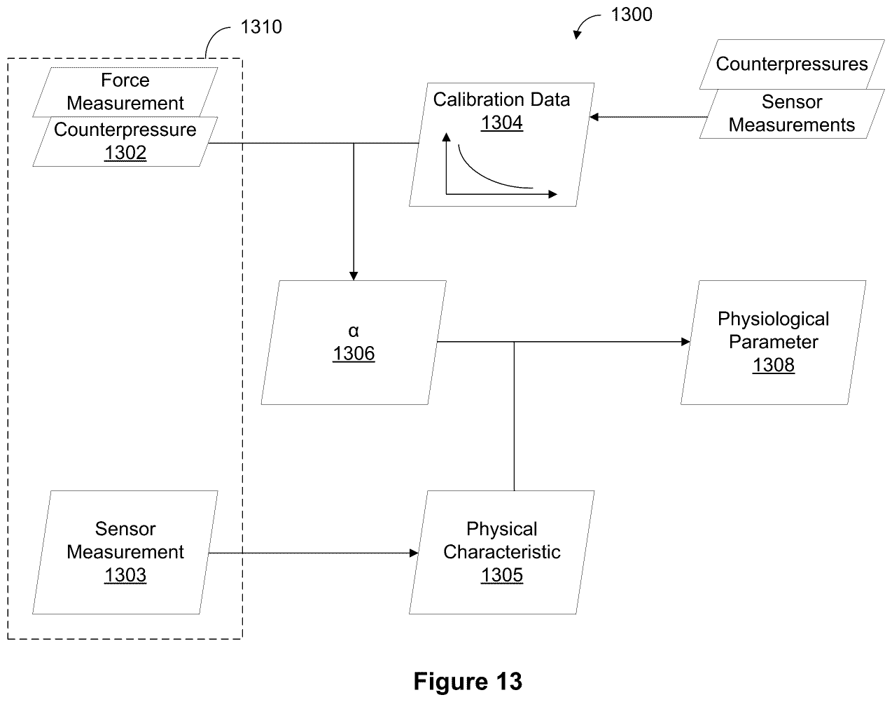

FIG. 13 is a block diagram that shows a flow diagram 1300 for determining a physiological parameter using measurements by a sensor apparatus and calibration data, which can be utilized in embodiments as described herein. In some embodiments, a sensing apparatus (e.g., apparatus 600, which may be or incorporate a handheld device such as a probe, or be or incorporate wearable device such as a wristband or a ring) may be configured to obtain a force measurement corresponding to an external pressure being applied by the sensing apparatus, via a force sensor (e.g., force sensing system 602, force sensor 702, force sensor 802, force sensor 902, force sensor 1002, force sensor 1102) as described elsewhere herein. External pressure (counterpressure 1302) may be determined based on the force measurement, e.g., based on an area of a sensing area and the force measurement as mentioned above. Calibration data 1304 may also be obtained, e.g., via approaches described above, including empirical observations and data collection (which may involve having acquired sensor measurements based on known counterpressures to determine certain physical characteristics such as cross-sectional area of a blood vessel, discussed above and below) and/or a ML model. For example, a look-up curve relating external counterpressure to a calibration factor α may be obtained for a user, such as curve 1204. In some approaches, a calibration factor (α) 1306 may be determined from the counterpressure 1302 and the calibration data 1304, e.g., according to Equation 1. Accurate force measurement ensures consistency between measurements and calibration data 1304.

In some embodiments, the sensing apparatus may be configured to obtain a sensor measurement 1303. The sensor measurement 1303 may be based on signals obtained from a target object (e.g., blood vessel) using one or more of various modalities, including PAPG, PEU, PPG, acoustics, IR, and/or RF as described above. In some examples, an ultrasonic response may be generated from the target object based on stimulation by optical signals (e.g., light from a light source of a PAPG sensor system) or acoustic signals (e.g., ultrasound pulses from a PEU sensor system). Returning ultrasonic waves may be detected using the PAPG and/or PeU sensor systems.

In some embodiments, at least one physical characteristic 1305 of the target object may be, for example, a cross-sectional area of a blood vessel (A_{sys.} and A_{dia.}). In some approaches, the physical characteristic 1305 (arterial cross-sectional area) may be determined based on the sensor measurement 1303.

In some embodiments, a physiological parameter of the user may be determined based on the physical characteristic 1305 and the calibration factor 1306. In some embodiments, the physiological parameter of the user may include an estimated blood pressure of the user at a given time. More specifically, cross-sectional area of the blood vessel at a given time (A₀) may be determined based on the physical characteristic 1305, and P(t) may be determined based on A₀ and the calibration factor 1306 (α) according to Equation 2. In some cases, a waveform may be determined for P(t) such that blood pressure can be estimated at any given time during operation of the sensing apparatus.

Advantageously, a non-invasive and cuffless method of estimating blood pressure may be enabled with cuffless and/or wearable form factors that do not apply significant external pressure to the user, such as a probe, ring, wristband, etc. in which a compact force sensor can be implemented internally or externally as discussed above.

### Example Methods

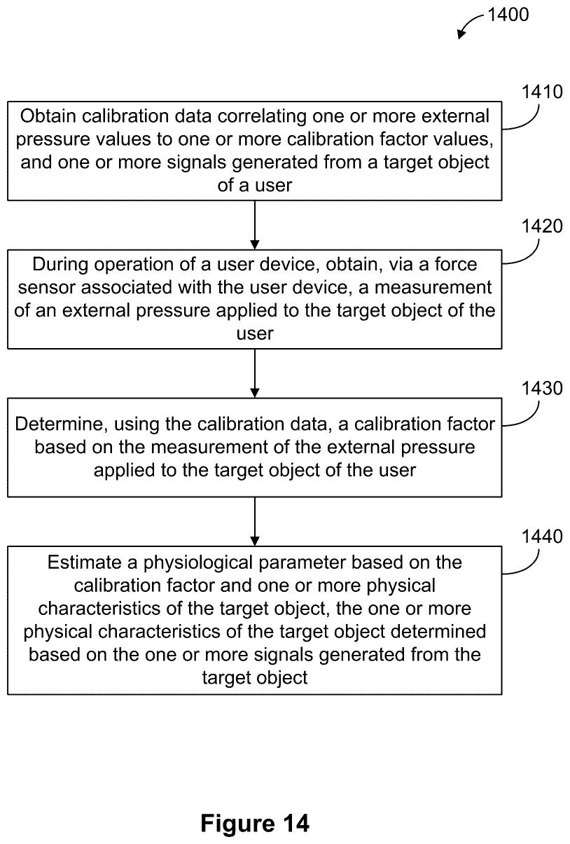

FIG. 14 is a flow diagram of a method 1400 of estimating a physiological parameter using a user device, according to some embodiments. Structure for performing the functionality illustrated in one or more of the blocks shown in FIG. 14 may be performed by hardware and/or software components of a sensor apparatus or system. Components of such sensor apparatus or system may include, for example, a force sensing system, an object sensing system, a control system (including one or more processors), a memory, and/or a computer-readable apparatus including a storage medium storing computer-readable and/or computer-executable instructions that are configured to, when executed by the control system, cause the control system, the one or more processors, and/or the sensor apparatus or system to perform operations represented by blocks below. Example components of the sensor apparatus or system are illustrated in, e.g., FIGS. 6-11, which are described in more detail above.

The blocks of FIG. 14 may, for example, be performed by the apparatus 600, the sensor apparatus 700, the sensor apparatus 800, the sensor apparatus 900, the sensor apparatus 1000, the sensor apparatus 1100, or by a similar apparatus, or a component thereof (e.g., control system 606). Implementation of the method described with respect to FIG. 14 may include more or fewer blocks than indicated. Moreover, the blocks of methods disclosed herein are not necessarily performed in the order indicated. Further, one or more of the blocks shown in FIG. 14 may be performed concurrently.

At block 1410, the method 1400 may include obtaining calibration data correlating one or more external pressure values to one or more calibration factor values, and obtaining one or more signals generated from a target object of a user. In some embodiments, the one or more signals generated from the target object of the user may include signals generated from emitted optical signals (e.g., light, IR), emitted acoustic signals (e.g., ultrasonic waves), emitted electromagnetic signals (e.g., RF), or a combination thereof incident on the target object.

In some embodiments, the obtaining of the calibration data may include applying a plurality of external pressure values; measuring one or more physical characteristics of the target object at each of the plurality of external pressure values; determining the one or more calibration factor values, each based on the one or more physical characteristics of the target object; and determining a relationship between the plurality of external pressure values and the one or more calibration factor values.

In some implementations, the target object may include a blood vessel of the user; the one or more physical characteristics of the target object comprise a cross-sectional area of the blood vessel during systole, a cross-sectional area of the blood vessel during diastole; the one or more calibration factor values may each be based on the cross-sectional area of the blood vessel during systole, the cross-sectional area of the blood vessel during diastole, a pressure of the blood vessel during systole, and a pressure of the blood vessel during diastole. In some implementations, the method 1400 may further include determining the one or more physical characteristics of the target object of the user based on the at least one or more acoustic signals generated from the target object of the user, the at least one or more acoustic signals comprising one or more photoacoustic signals generated from the blood vessel, one or more acoustic signals generated from the blood vessel, or a combination thereof.

Means for performing functionality at block 1410 may comprise a control system 606, a communication interface system 608, and/or other components of a sensor apparatus, as illustrated in FIGS. 6-11.

At block 1420, the method 1400 may include, during operation of the user device, obtaining, via a force sensor associated with the user device, a measurement of an external pressure applied to the target object of the user.

In some embodiments, the force sensor associated with the user device may be disposed internally to the user device and configured to detect force associated with the external pressure via an interface of the user device. In some embodiments, the force sensor associated with the user device may be disposed externally to the user device such that the user device is disposed between the force sensor and the target object (an example of which is shown in FIG. 10), or such that the target object is disposed between the user device and the force sensor (an example of which is shown in FIG. 11).

In some embodiments, the user device may include a signal source system configured to emit optical signals, acoustic signals, electromagnetic signals, or a combination thereof toward a target object; and a sensor system configured to detect signals generated from the emitted optical signals, the emitted acoustic signals, the emitted electromagnetic signals, or the combination thereof incident on the target object. In some embodiments, the user device may further include an interface portion contactable by a user; and a force sensor system configured to measure an external pressure applied to the interface portion during contact by the user. In some implementations, the interface portion may be contactable by a user at a first side of a surface of the user device, and the force sensor system may be disposed at a second side of the surface of the user device. In some embodiments, the user device may further include a control system configured to perform the method 1400, including in some cases, obtain calibration data correlating one or more external pressure values to one or more calibration factor values; determine, using the calibration data, a calibration factor based at least on the external pressure applied to the interface portion during the contact by the user; and estimate a physiological parameter based at least on the calibration factor, and one or more physical characteristics of the target object of the user determined based on the signals detected by the sensor system.

In some implementations, the signal source system may include a light source system configured to generate and emit the optical signals toward the blood vessel of the user; and the sensor system may include a photoacoustic sensor system configured to obtain photoacoustic signals generated from the emitted optical signals incident on the blood vessel of the user, a pulse echo ultrasound (PeU) sensor system configured to obtain ultrasonic waves generated from the emitted acoustic signals incident on the blood vessel of the user, or a combination thereof.

In some implementations, the signal source system may include a microphone configured to obtain acoustic signals generated from pulsatile blood flow in blood vessel of the user.

In some embodiments, the user device may further include a housing. In some implementations, the first side of the surface of the user device may include an outwardly facing side of the housing, and the second side of the surface of the user device may include an internally facing side of the housing.

In some implementations, the force sensor system may be disposed along an axis orthogonal to the interface portion. In some implementations, the force sensor system may be disposed adjacent to the sensor system.

In some implementations, the signal source system may be disposed adjacent to the sensor system, the force sensor system, or both.

In some embodiments, the external pressure may be measured based on a magnitude of a force applied to a sensing portion of the interface portion, and an area of the sensing portion.

In some embodiments, the force sensor system may be disposed externally to a housing of a system. In some implementations, the force sensor system may be disposed externally to the housing such that the housing is disposed between the force sensor system and the target object, or such that the target object is disposed between the housing and the force sensor system.

Means for performing functionality at block 1420 may comprise an interface 601, a force sensing system 602, and/or other components of a sensor apparatus, as illustrated in FIGS. 6-11.

At block 1430, the method 1400 may include determining, using the calibration data, a calibration factor based on the measurement of the external pressure applied to the target object of the user.

In some embodiments, the calibration factor may be based on a cross-sectional area of the blood vessel during systole, a cross-sectional area of the blood vessel during diastole, a pressure of the blood vessel during systole, and a pressure of the blood vessel during diastole.

Means for performing functionality at block 1430 may comprise a force sensing system 602, and/or other components of a sensor apparatus, as illustrated in FIGS. 6-11.

At block 1440, the method 1400 may include estimating the physiological parameter based on the calibration factor and one or more physical characteristics of the target object, the one or more physical characteristics of the target object determined based on the one or more signals generated from the target object.

In some embodiments, the estimated physiological parameter may include a blood pressure of the user.

In some embodiments, the one or more physical characteristics may include a cross-sectional area of the blood vessel in a zero external pressure state, a cross-sectional area of the blood vessel at a given time, or a combination thereof.

Means for performing functionality at block 1440 may comprise an object sensing system 604, a control system 606, and/or other components of a sensor apparatus, as illustrated in FIGS. 6-11.

It will be apparent to those skilled in the art that substantial variations may be made in accordance with specific requirements. For example, customized hardware might also be used and/or particular elements might be implemented in hardware, software (including portable software, such as applets, etc.), or both. Further, connection to other computing devices such as network input/output devices may be employed.

Additionally, certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. Moreover, various ones of the described and illustrated operations can itself include and collectively refer to a number of sub-operations. For example, each of the operations described above can itself involve the execution of a process or algorithm. Furthermore, various ones of the described and illustrated operations can be combined or performed in parallel in some implementations. Similarly, the separation of various system components in the implementations described above should not be understood as requiring such separation in all implementations. As such, other implementations are within the scope of the following claims. In some cases, the actions recited in the claims can be performed in a different order and still achieve desirable results.

It will be understood that unless features in any of the particular described implementations are expressly identified as incompatible with one another or the surrounding context implies that they are mutually exclusive and not readily combinable in a complementary and/or supportive sense, the totality of this disclosure contemplates and envisions that specific features of those complementary implementations may be selectively combined to provide one or more comprehensive, but slightly different, technical solutions. It will therefore be further appreciated that the above description has been given by way of example only and that modifications in detail may be made within the scope of this disclosure.

With reference to the appended figures, components that can include memory can include non-transitory machine-readable media. The term “machine-readable medium” and “computer-readable medium” as used herein, refer to any storage medium that participates in providing data that causes a machine to operate in a specific fashion. In embodiments provided hereinabove, various machine-readable media might be involved in providing instructions/code to processors and/or other device(s) for execution. Additionally or alternatively, the machine-readable media might be used to store and/or carry such instructions/code. In many implementations, a computer-readable medium is a physical and/or tangible storage medium. Such a medium may take many forms, including but not limited to, non-volatile media and volatile media. Common forms of computer-readable media include, for example, magnetic and/or optical media, any other physical medium with patterns of holes, a RAM, a programmable ROM (PROM), erasable PROM (EPROM), a FLASH-EPROM, any other memory chip or cartridge, or any other medium from which a computer can read instructions and/or code.

The methods, systems, and devices discussed herein are examples. Various embodiments may omit, substitute, or add various procedures or components as appropriate. For instance, features described with respect to certain embodiments may be combined in various other embodiments. Different aspects and elements of the embodiments may be combined in a similar manner. The various components of the figures provided herein can be embodied in hardware and/or software. Also, technology evolves and, thus many of the elements are examples that do not limit the scope of the disclosure to those specific examples.

It has proven convenient at times, principally for reasons of common usage, to refer to such signals as bits, information, values, elements, symbols, characters, variables, terms, numbers, numerals, or the like. It should be understood, however, that all of these or similar terms are to be associated with appropriate physical quantities and are merely convenient labels. Unless specifically stated otherwise, as is apparent from the discussion above, it is appreciated that throughout this Specification discussion utilizing terms such as “processing,” “computing,” “calculating,” “determining,” “ascertaining,” “identifying,” “associating,” “measuring,” “performing,” or the like refer to actions or processes of a specific apparatus, such as a special purpose computer or a similar special purpose electronic computing device. In the context of this Specification, therefore, a special purpose computer or a similar special purpose electronic computing device is capable of manipulating or transforming signals, typically represented as physical electronic, electrical, or magnetic quantities within memories, registers, or other information storage devices, transmission devices, or display devices of the special purpose computer or similar special purpose electronic computing device.

Terms, “and” and “or” as used herein, may include a variety of meanings that also is expected to depend, at least in part, upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein may be used to describe any feature, structure, or characteristic in the singular or may be used to describe some combination of features, structures, or characteristics. However, it should be noted that this is merely an illustrative example and claimed subject matter is not limited to this example. Furthermore, the term “at least one of” if used to associate a list, such as A, B, or C, can be interpreted to mean any combination of A, B, and/or C, such as A, AB, AA, AAB, AABBCCC, etc.

Having described several embodiments, various modifications, alternative constructions, and equivalents may be used without departing from the scope of the disclosure. For example, the above elements may merely be a component of a larger system, wherein other rules may take precedence over or otherwise modify the application of the various embodiments. Also, a number of steps may be undertaken before, during, or after the above elements are considered. Accordingly, the above description does not limit the scope of the disclosure.

In view of this description embodiments may include different combinations of features. Implementation examples are described in the following numbered clauses:
Clause 1. A user device comprising: a signal source system configured to emit optical signals, acoustic signals, electromagnetic signals, or a combination thereof toward a target object; a sensor system configured to detect signals generated from the emitted optical signals, the emitted acoustic signals, the emitted electromagnetic signals, or the combination thereof incident on the target object; an interface portion contactable by a user; a force sensor system configured to measure an external pressure applied to the interface portion during contact by the user; and a control system configured to: obtain calibration data correlating one or more external pressure values to one or more calibration factor values; determine, using the calibration data, a calibration factor based on the external pressure applied to the interface portion during the contact by the user; and estimate a physiological parameter based on the calibration factor, and one or more physical characteristics of the target object of the user determined based on the signals detected by the sensor system.
Clause 2. The user device of clause 1, wherein the estimated physiological parameter comprises a blood pressure of the user.
Clause 3. The user device of clause 1, wherein the control system is further configured to, to obtain the calibration data: apply a plurality of external pressure values; measure one or more physical characteristics of the target object at each of the plurality of external pressure values; determine the one or more calibration factor values, each based on the one or more physical characteristics of the target object; and determine a relationship between the plurality of external pressure values and the one or more calibration factor values.
Clause 4. The user device of clause 1, wherein: the target object comprises a blood vessel of the user; the signal source system comprises a light source system configured to generate and emit the optical signals toward the blood vessel of the user; and the sensor system comprises a photoacoustic sensor system configured to obtain photoacoustic signals generated from the emitted optical signals incident on the blood vessel of the user, a pulse echo ultrasound (PeU) sensor system configured to obtain ultrasonic waves generated from the emitted acoustic signals incident on the blood vessel of the user, or a combination thereof.
Clause 5. The user device of clause 1, wherein: the target object comprises a blood vessel of the user; and the signal source system comprises a microphone configured to obtain acoustic signals generated from pulsatile blood flow in blood vessel of the user.
Clause 6. The user device of clause 1, further comprising a housing; wherein the interface portion may be contactable by a user at an outwardly facing surface of the housing, and the force sensor system may be disposed at an internally facing side of the surface of the housing.
Clause 7. The user device of clause 1, wherein the force sensor system is disposed along an axis orthogonal to the interface portion.
Clause 8. The user device of clause 1, wherein the force sensor system is disposed adjacent to the sensor system.
Clause 9. The user device of clause 1, wherein: the target object comprises a blood vessel of the user; and the one or more physical characteristics comprise a cross-sectional area of the blood vessel in a zero external pressure state, a cross-sectional area of the blood vessel at a given time, or a combination thereof.
Clause 10. The user device of clause 1, wherein: the target object comprises a blood vessel of the user; and the calibration factor is based on a cross-sectional area of the blood vessel during systole, a cross-sectional area of the blood vessel during diastole, a pressure of the blood vessel during systole, and a pressure of the blood vessel during diastole.
Clause 11. The user device of clause 1, wherein the signal source system is disposed adjacent to the sensor system, the force sensor system, or both.
Clause 12. The user device of clause 1, wherein the external pressure is measured based on a magnitude of a force applied to a sensing portion of the interface portion, and an area of the sensing portion.
Clause 13. A system configured to estimate a physiological parameter, the system comprising: a housing comprising: a signal source system configured to emit optical signals, acoustic signals, electromagnetic signals, or a combination thereof toward a target object; a sensor system configured to detect acoustic signals generated from the emitted optical signals, the emitted acoustic signals, the emitted electromagnetic signals, or the combination thereof incident on the target object; and an interface portion contactable by a user; a force sensor system disposed externally to the housing, the force sensor system configured to measure an external pressure applied during contact by the user; and a control system configured to: obtain calibration data correlating one or more external pressure values to one or more calibration factor values; determine, using the calibration data, a calibration factor based at least on the external pressure during the contact by the user; and estimate a physiological parameter based at least on the calibration factor and one or more physical characteristics of the target object of the user determined based on the acoustic signals detected by the sensor system.
Clause 14. The system of clause 13, wherein the force sensor system is disposed externally to the housing such that the housing is disposed between the force sensor system and the target object, or such that the target object is disposed between the housing and the force sensor system.
Clause 15. A method of estimating a physiological parameter using a user device, the method comprising: obtaining calibration data correlating one or more external pressure values to one or more calibration factor values, and obtaining one or more signals generated from a target object of a user; during operation of the user device, obtaining, via a force sensor associated with the user device, a measurement of an external pressure applied to the target object of the user; determining, using the calibration data, a calibration factor based on the measurement of the external pressure applied to the target object of the user; and estimating the physiological parameter based on the calibration factor and one or more physical characteristics of the target object, the one or more physical characteristics of the target object determined based on the one or more signals generated from the target object.
Clause 16. The method of clause 15, wherein the estimated physiological parameter comprises a blood pressure of the user.
Clause 17. The method of clause 15, wherein the force sensor associated with the user device is disposed internally to the user device and configured to detect force associated with the external pressure via an interface of the user device.
Clause 18. The method of clause 15, wherein the force sensor associated with the user device is disposed externally to the user device such that the user device is disposed between the force sensor and the target object, or such that the target object is disposed between the user device and the force sensor.
Clause 19. The method of clause 15, wherein the obtaining of the calibration data comprises: applying a plurality of external pressure values; measuring one or more physical characteristics of the target object at each of the plurality of external pressure values; determining the one or more calibration factor values, each based on the one or more physical characteristics of the target object; and determining a relationship between the plurality of external pressure values and the one or more calibration factor values.
Clause 20. The method of clause 19, wherein: the target object comprises a blood vessel of the user; the one or more physical characteristics of the target object comprise a cross-sectional area of the blood vessel during systole, a cross-sectional area of the blood vessel during diastole; the one or more calibration factor values is each be based on the cross-sectional area of the blood vessel during systole, the cross-sectional area of the blood vessel during diastole, a pressure of the blood vessel during systole, and a pressure of the blood vessel during diastole; and the method further comprises determining the one or more physical characteristics of the target object of the user based on one or more acoustic signals generated from the target object of the user, the one or more acoustic signals comprising one or more photoacoustic signals generated from the blood vessel, one or more acoustic signals generated from the blood vessel, or a combination thereof.

1. A user device comprising:
a signal source system configured to emit optical signals, acoustic signals, electromagnetic signals, or a combination thereof toward a target object;
a sensor system configured to detect signals generated from the emitted optical signals, the emitted acoustic signals, the emitted electromagnetic signals, or the combination thereof incident on the target object;
an interface portion contactable by a user;
a force sensor system configured to measure an external pressure applied to the interface portion during contact by the user; and
a control system configured to:
obtain calibration data correlating one or more external pressure values to one or more calibration factor values;
determine, using the calibration data, a calibration factor based on the external pressure applied to the interface portion during the contact by the user; and
estimate a physiological parameter based on the calibration factor, and one or more physical characteristics of the target object of the user determined based on the signals detected by the sensor system.

2. The user device of claim 1, wherein the estimated physiological parameter comprises a blood pressure of the user.

3. The user device of claim 1, wherein the control system is further configured to, to obtain the calibration data:
apply a plurality of external pressure values;
measure one or more physical characteristics of the target object at each of the plurality of external pressure values;
determine the one or more calibration factor values, each based on the one or more physical characteristics of the target object; and
determine a relationship between the plurality of external pressure values and the one or more calibration factor values.

4. The user device of claim 1, wherein:
the target object comprises a blood vessel of the user;
the signal source system comprises a light source system configured to generate and emit the optical signals toward the blood vessel of the user; and
the sensor system comprises a photoacoustic sensor system configured to obtain photoacoustic signals generated from the emitted optical signals incident on the blood vessel of the user, a pulse echo ultrasound (PeU) sensor system configured to obtain ultrasonic waves generated from the emitted acoustic signals incident on the blood vessel of the user, or a combination thereof.

5. The user device of claim 1, wherein:
the target object comprises a blood vessel of the user; and
the signal source system comprises a microphone configured to obtain acoustic signals generated from pulsatile blood flow in blood vessel of the user.

6. The user device of claim 1, further comprising a housing;
wherein the interface portion may be contactable by a user at an outwardly facing surface of the housing, and the force sensor system may be disposed at an internally facing side of the surface of the housing.

7. The user device of claim 1, wherein the force sensor system is disposed along an axis orthogonal to the interface portion.

8. The user device of claim 1, wherein the force sensor system is disposed adjacent to the sensor system.

9. The user device of claim 1, wherein:
the target object comprises a blood vessel of the user; and
the one or more physical characteristics comprise a cross-sectional area of the blood vessel in a zero external pressure state, a cross-sectional area of the blood vessel at a given time, or a combination thereof.

10. The user device of claim 1, wherein:
the target object comprises a blood vessel of the user; and
the calibration factor is based on a cross-sectional area of the blood vessel during systole, a cross-sectional area of the blood vessel during diastole, a pressure of the blood vessel during systole, and a pressure of the blood vessel during diastole.

11. The user device of claim 1, wherein the signal source system is disposed adjacent to the sensor system, the force sensor system, or both.

12. The user device of claim 1, wherein the external pressure is measured based on a magnitude of a force applied to a sensing portion of the interface portion, and an area of the sensing portion.

13. A system configured to estimate a physiological parameter, the system comprising:
a housing comprising:
a signal source system configured to emit optical signals, acoustic signals, electromagnetic signals, or a combination thereof toward a target object;
a sensor system configured to detect acoustic signals generated from the emitted optical signals, the emitted acoustic signals, the emitted electromagnetic signals, or the combination thereof incident on the target object; and
an interface portion contactable by a user;
a force sensor system disposed externally to the housing, the force sensor system configured to measure an external pressure applied during contact by the user; and
a control system configured to:
obtain calibration data correlating one or more external pressure values to one or more calibration factor values;
determine, using the calibration data, a calibration factor based at least on the external pressure during the contact by the user; and
estimate a physiological parameter based at least on the calibration factor and one or more physical characteristics of the target object of the user determined based on the acoustic signals detected by the sensor system.

14. The system of claim 13, wherein the force sensor system is disposed externally to the housing such that the housing is disposed between the force sensor system and the target object, or such that the target object is disposed between the housing and the force sensor system.

15. A method of estimating a physiological parameter using a user device, the method comprising:
obtaining calibration data correlating one or more external pressure values to one or more calibration factor values, and obtaining one or more signals generated from a target object of a user;
during operation of the user device, obtaining, via a force sensor associated with the user device, a measurement of an external pressure applied to the target object of the user;
determining, using the calibration data, a calibration factor based on the measurement of the external pressure applied to the target object of the user; and
estimating the physiological parameter based on the calibration factor and one or more physical characteristics of the target object, the one or more physical characteristics of the target object determined based on the one or more signals generated from the target object.

16. The method of claim 15, wherein the estimated physiological parameter comprises a blood pressure of the user.

17. The method of claim 15, wherein the force sensor associated with the user device is disposed internally to the user device and configured to detect force associated with the external pressure via an interface of the user device.

18. The method of claim 15, wherein the force sensor associated with the user device is disposed externally to the user device such that the user device is disposed between the force sensor and the target object, or such that the target object is disposed between the user device and the force sensor.

19. The method of claim 15, wherein the obtaining of the calibration data comprises:
applying a plurality of external pressure values;
measuring one or more physical characteristics of the target object at each of the plurality of external pressure values;
determining the one or more calibration factor values, each based on the one or more physical characteristics of the target object; and
determining a relationship between the plurality of external pressure values and the one or more calibration factor values.

20. The method of claim 19, wherein:
the target object comprises a blood vessel of the user;
the one or more physical characteristics of the target object comprise a cross-sectional area of the blood vessel during systole, a cross-sectional area of the blood vessel during diastole;
the one or more calibration factor values is each be based on the cross-sectional area of the blood vessel during systole, the cross-sectional area of the blood vessel during diastole, a pressure of the blood vessel during systole, and a pressure of the blood vessel during diastole; and
the method further comprises determining the one or more physical characteristics of the target object of the user based on one or more acoustic signals generated from the target object of the user, the one or more acoustic signals comprising one or more photoacoustic signals generated from the blood vessel, one or more acoustic signals generated from the blood vessel, or a combination thereof.
